(19) United States
(12) Patent Application Publication
(10) Pub. No.: US 2005/0201975 A1
(43) Pub. Date: Sep. 15, 2005

(54) Medical treatment

(76) Inventors: Brian Robert Champion, Cambridge (GB); Francesco Falciani, Birmingham (GB); Penelope Caroline Hayward, Cambridge (GB); Gareth Llewellyn Maslen, Cambridge (GB)

Correspondence Address: FROMMER LAWRENCE & HAUG, 745 FIFTH AVENUE- 10TH FL., NEW YORK, NY, 10151, US

(21) Appl. No.: 10/899,422

(22) Filed: Jul. 26, 2004

Related U.S. Application Data
(63) Continuation-in-part of application No. PCT/GB03/00303, filed on Jan. 27, 2003.

(30) Foreign Application Priority Data
Jan. 25, 2002 (GB) 0201674.9

(51) Int. Cl. C12Q 1/68; G01N 33/567; C07H 21/04; C12N 15/09; A61K 48/00; A61K 38/19; A61K 38/20; C07K 14/52

(52) U.S. Cl. 424/85.1; 424/85.2; 530/351; 435/6; 435/69.5; 435/320.1; 435/325; 536/23.5; 514/44

(57) ABSTRACT

A method is described for modifying chemokine signalling by administering an effective amount of a modulator of the Notch signalling pathway. Human homologues of certain proteins, polypeptides and polynucleotides involved in Notch and/or chemokine signalling pathways are also described.

### CROSS-REFERENCE TO RELATED APPLICATIONS

This application is a continuation-in-part of International Application No. PCT/GB03/00303, filed on Jan. 27, 2003, published as WO 03/062273 on Jul. 31, 2003, and claiming priority to GB application Serial No. 0201674.9, filed on Jan. 25, 2002. Reference is made to U.S. application Ser. No. 09/310,685, filed on May 4, 1999, Ser. No. 09/870,902, filed on May 31, 2001, Ser. No. 10/013,310, filed on Dec. 7, 2001, Ser. No. 10/147,354, filed on May 16, 2002, Ser. No. 10/357,321, filed on Feb. 3, 2002, Ser. No. 10/682,230, filed on Oct. 9, 2003, Ser. No. 10/720,896, filed on Nov. 24, 2003, Ser. Nos. 10/763,362, 10/764,415 and 10/765,727, all filed on Jan. 23, 2004, Ser. No. 10/812,144, filed on Mar. 29, 2004 and 10/845,834, filed on May 14, 2004. Reference is also made to International Application No. PCT/GB02/05133, filed on Nov. 13, 2002, and published as WO 03/042246 on May 22, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates inter alia to Notch signalling and particularly, but not exclusively, the effect of Notch signalling on chemokine signalling. The invention also provides inter alia human homologues of certain proteins, polypeptides and polynucleotides involved in Notch and/or chemokine signalling pathways.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising other epitopes on the same or related antigens, a phenomenon termed “epitope spreading”.

A description of the Notch signalling pathway and conditions affected by it may be found in our published PCT Applications WO 98/20142, WO 00/36089 and WO 0135990. The text of each of PCT/GB97/03058 (WO 98/20142), PCT/GB99/04233 (WO 00/36089) and PCT/GBOO/04391 (WO 0135990) is hereby incorporated herein by reference (see also Hoyne G. F. et al (1999) Int Arch Allergy Immunol 118:122-124; Hoyne et al. (2000) Immunology 100:281-288; Hoyne G. F. et al (2000) Intl Immunol 12:177-185; Hoyne, G. et al. (2001) Immunological Reviews 182:215-227 which are also incorporated by reference).

Notch ligand expression also plays a role in cancer. Indeed, upregulated Notch ligand expression has been observed in some tumour cells. These tumour cells are capable of rendering T cells unresponsive to restimulation with a specific antigen, thus providing a possible explanation of how tumour cells prevent normal T cell responses. By downregulating Notch signalling in vivo in T cells, it may be possible to prevent tumour cells from inducing immunotolerance in those T cells that recognise tumour-specific antigens. In turn, this would allow the T cells to mount an immune response against the tumour cells (WO00/135990).

Without wishing to be bound by any theory or mechanism of action, in one aspect of the present invention it has been found that the Notch intracellular domain (Notch IC) interacts with a protein similar to mouse C2PA (Linares et al, FEBS Lett, Vol 480, 249-254, September 2000) and PDZ-RGS3 (Lu et al, Cell, Vol 105, 66-79, April 2001). Similar such proteins are known, for example, from Lu et al supra and Reif and Cyster (The Journal of Immunology, 2000, 164, 4720-4729) to modify chemoattraction/chemokine signalling.

As described, for example, in U.S. Pat. No. 6,323,206, chemoattractant cytokines or chemokines are a family of proinflammatory mediators that promote recruitment and activation of multiple lineages of leukocytes and lymphocytes. They can be released by many kinds of tissue cells after activation. Continuous release of chemokines at sites of inflammation mediates the ongoing migration of effector cells in chronic inflammation. The chemokines characterized to date are related in primary structure. They share four conserved cysteines, which form disulfide bonds. Based upon this conserved cysteine motif, the family is divided into two main branches, designated as the C—X—C chemokines (alpha-chemokines), and the C—C chemokines (beta-chemokines), in which the first two conserved cysteines are separated by an intervening residue, or adjacent respectively (Baggiolini, M. and Dahinden, C. A., Immunology Today, 15:127-133 (1994)).

The C—X—C chemokines include a number of potent chemoattractants and activators of neutrophils, such as interleukin 8 (IL-8), PF4 and neutrophil-activating peptide-2 (NAP-2). The C—C chemokines include RANTES (Regulated on Activation, Normal T Expressed and Secreted), the macrophage inflammatory proteins 1.alpha. and 1.beta. (MIP-1 alpha and MIP-1beta), and human monocyte chemotatic proteins 1-3 (MCP-1, MCP-2, MCP-3), which have been characterized as chemoattractants and activators of monocytes or lymphocytes but do not appear to be chemoattractants for neutrophils. Chemokines, such as RANTES and MIP-1 alpha, have been implicated in a wide range of human acute and chronic inflammatory diseases including respiratory diseases, such as asthma and allergic disorders.

### SUMMARY OF THE INVENTION

According to a first aspect of the invention there is provided a method for modifying chemokine signalling by administering an effective amount of a modulator of the Notch signalling pathway.

According to a further aspect of the invention there is provided a method for modifying chemokine signal transduction by administering an effective amount of a modulator of the Notch signalling pathway.

Without wishing to be bound by any theory of mode of action, it is believed that Notch interacts inter alia with PDZ-RGS proteins which modify chemokine signalling, possibly but modifying (enhancing or inhibiting) G-protein signal transduction from the chemokine receptor. Notch signalling has also been shown by the present inventors to increase chemokine expression.

In a further aspect, therefore, there is provided a method for modifying chemokine receptor signalling by administering an effective amount of a modulator of the Notch signalling pathway.

According to a further aspect of the invention there is provided a method for treating inflammation by administering an effective amount of a modulator of the Notch signalling pathway.

In one embodiment of the invention chemokine signalling and/or signal transduction may be increased or enhanced. In an alternative embodiment chemokine signalling may be decreased.

In one embodiment of the invention the modulator of the Notch signalling pathway may be an activator of Notch signalling, and suitably a Notch receptor agonist. In an alternative embodiment the modulator of the Notch signalling pathway may be an inhibitor of Notch signalling, suitably a Notch receptor antagonist.

Whether an agent is an activator or inhibitor of Notch signalling may be determined by use of any suitable screen or assay, for example, an ELISA assay as described in Example 9 herein, or a reporter assay as described, for example, in Shimizu et al, Mol Cell Biol 2000 September; 20 (18): 6913-22 and/or in our co-pending International Application PCT/GB2002/03397.

According to a further aspect of the invention there is provided a method for treating a disease associated with leukocyte recruitment and/or activation mediated by chemokine function comprising administering an effective amount of a modulator of the Notch signalling pathway.

According to a further aspect of the invention there is provided the use of a modulator of the Notch signalling pathway in the manufacture of a medic ament for modifying chemokine signalling. Suitably the chemokine signalling may be SDF1 signalling.

According to a further aspect of the invention there is provided the use of a modulator of the Notch signalling pathway in the manufacture of a medicament for modifying chemokine signal transduction.

According to a further aspect of the invention there is provided the use of a modulator of the Notch signalling pathway in the manufacture of a medicament for the treatment of a disease associated with leukocyte recruitment and/or activation mediated by chemokine function.

According to a further aspect of the invention there is provided the use of a modulator of the Notch signalling pathway to modify cell migration, such as leukocyte chemotaxis and extravasion, for example in inflammatory processes, or in developmental/repair processes such as axonal development, neuronal migration or angiogenesis.

In one embodiment such migration, chemotaxis or extravasion may be increased. In an alternative embodiment such migration, chemotaxis or extravasion may be decreased.

According to a further aspect of the invention there is provided the use of a modulator of the Notch signalling pathway in the manufacture of a medicament for the treatment of inflammation.

Preferably the modulator of the Notch signalling pathway may comprise a fusion protein or a polynucleotide which codes for a fusion protein. For example, the modulator may be a fusion protein comprising a segment of a Notch ligand extracellular domain and an immunoglobulin Fc segment or a polynucleotide encoding such a fusion protein.

Suitably the modulator of the Notch signalling pathway comprises a protein or polypeptide comprising a DSL domain and at least one EGF-like domain or a fragment, derivative, homologue, analogue or allelic variant thereof or a polynucleotide sequence coding for such a protein, polypeptide, fragment, derivative, homologue, analogue or allelic variant.

Alternatively or in addition the modulator of the Notch signalling pathway may comprise Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

Suitably the modulator of the Notch signalling pathway comprises Delta or a fragment, derivative, homologue, analogue or allelic variant thereof or a polynucleotide encoding Delta or a fragment, derivative, homologue, analogue or allelic variant thereof.

Alternatively or in addition the modulator of the Notch signalling pathway may comprise Serrate/Jagged or a fragment, derivative, homologue, analogue or allelic variant thereof or a polynucleotide encoding Serrate/Jagged or a fragment, derivative, homologue, analogue or allelic variant thereof.

Alternatively or in addition the modulator of the Notch signalling pathway may comprise Notch or a fragment, derivative, homologue, analogue or allelic variant thereof or a polynucleotide encoding Notch or a fragment, derivative, homologue, analogue or allelic variant thereof.

Alternatively or in addition the modulator of the Notch signalling pathway may comprise a dominant negative version of a Notch signalling repressor, or a polynucleotide which codes for a dominant negative version of a Notch signalling repressor.

Alternatively or in addition the modulator of the Notch signalling pathway may comprise a polypeptide capable of upregulating the expression or activity of a Notch ligand or a downstream component of the Notch signalling pathway, or a polynucleotide which codes for such a polypeptide.

Alternatively or in addition the modulator of the Notch signalling pathway may comprise a polypeptide selected from Noggin, Chordin, Follistatin, Xnr3 and FGF or a fragment, derivative, homologue, analogue or allelic variant thereof, or a polynucleotide which codes for such a polypeptide, fragment, derivative, homologue, analogue or allelic variant.

Alternatively or in addition the modulator of the Notch signalling pathway may comprise an immunosuppressive cytokine selected from IL-4, IL-10, IL-13, TGF-β and FLT3 ligand or a fragment, derivative, homologue, analogue or allelic variant thereof, or a polynucleotide which codes for such an immunosuppressive cytokine, fragment, derivative, homologue, analogue or allelic variant.

Alternatively, the modulator of the Notch signalling pathway may comprise an antibody, antibody fragment or antibody derivative or a polynucleotide which codes for an antibody, antibody fragment or antibody derivative.

According to a further aspect of the invention there is provided a method of modulating the trafficking of cells such as leukocytes, the method comprising: administering an effective amount of a modulator of Notch signalling, in a dose effective to modulate said trafficking of said cells.

According to a further aspect of the invention there is provided a method of enhancing migration of cells by administering a modulator of Notch signalling in an amount effective to enhance migration of cells.

According to a further aspect of the invention there is provided a method of enhancing migration of cells to a site in a subject comprising locally administering to said site a modulator of Notch signalling in an amount effective to enhance migration of cells to the site in the subject.

According to a further aspect of the invention there is provided a method of inhibiting migration of cells comprising administering, suitably locally administering, a modulator of Notch signalling in an amount effective to inhibit migration of cells.

According to a further aspect of the invention there is provided a method of inhibiting migration of cells to a site in a subject comprising locally administering to said site a modulator of Notch signalling in an amount effective to inhibit migration of cells to the site in the subject.

Suitably the cells whose migration is modified may be immune cells, neural cells, epithelial cells, endothelial cells or mesenchymal cells.

According to a further aspect, modulators of Notch signalling or proteins, polypeptides or polynucleotides according to the present invention may be applied to implants and prostheses for use in medicine to increase or decrease cells for the surface thereof.

According to a further aspect of the invention therefore there is provided a method of repelling cells from a material surface comprising coating a material surface with an amount of a modulator of Notch signalling effective to repel cells from the material surface.

According to a further aspect of the invention there is provided a method of attracting cells to a material surface comprising coating a material surface with a modulator of Notch signalling in an effective amount to attract cells to the material surface.

The invention further provides an implant or prosthesis coated with a modulator of Notch signalling or a protein, polypeptide or polynucleotide according to the present invention.

According to a further aspect of the invention there is provided a method of enhancing an immune response in a subject having a condition that involves defective chemokine signalling.

According to a further aspect of the invention there is provided a method for inhibiting angiogenesis by administering a modulator of Notch signalling.

According to a further aspect of the invention there is provided a method for increasing angiogenesis by administering a modulator of Notch signalling.

According to a further aspect of the invention there is provided a method for modifying ephrin signalling by administering an effective amount of a modulator of the Notch signalling pathway. Ephrin signalling may be either increased or decreased according to the invention.

According to a further aspect of the invention there is provided a method of altering the sensitivity of a cell to a chemokine by administering a modulator of Notch signalling.

Suitably such methods may be used for modifying cell migration, blood vessel formation, or axon pathway selection.

According to a further aspect of the invention there is provided a method for modifying cell migration by administering an effective amount of a modulator of the Notch signalling pathway.

According to a further aspect of the invention there is provided a method for modifying cellular extravasion by administering an effective amount of a modulator of the Notch signalling pathway.

Proteins, polypeptides and polynucleotides comprising or coding for PDZ and/or RGS domains may be used to modify chemoattraction, as described, for example in WO 02/079382 (President and Fellows of Harvard College). The human proteins, polypeptides and polynucleotides provided herein may therefore be used inter alia to treat conditions as described infra under the heading “Therapeutic Uses” and as targets for screening of modulators.

According to a further aspect of the invention there is provided a polynucleotide comprising a nucleotide sequence which has at least 50%, preferably at least 70% homology or identity (preferably at least 80%, 85% 90%, 95% or 99% homology or identity) to the sequence of SEQ ID NO: 5, preferably over the entire length of SEQ ID NO: 5, or a nucleotide sequence fully complementary to said polynucleotide (see FIG. 5).

According to a further aspect of the invention there is provided a polynucleotide having the sequence of SEQ ID NO: 5.

According to a further aspect of the invention there is provided a polypeptide comprising an amino acid sequence which has at least 70% identity (preferably at least 80%, preferably at least 85%, preferably at least 90%, preferably at least 95% or at least 99% identity) to the amino acid sequence of SEQ ID NO: 6, preferably over the entire length of the latter (see FIG. 5).

According to a further aspect of the invention there is provided a polypeptide comprising the amino acid sequence of SEQ ID NO: 6.

According to a further aspect of the invention there is provided a polynucleotide comprising a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 6, preferably over the entire length of SEQ ID NO: 6, or a nucleotide sequence fully complementary to said polynucleotide.

According to a further aspect of the invention there is provided a polynucleotide having the sequence of SEQ ID NO: 6.

According to a further aspect of the invention there is provided a polypeptide comprising an amino acid sequence which has at least 70% identity (preferably at least 80%, preferably at least 85%, preferably at least 90%, preferably at least 95% or at least 99% identity) to the amino acid sequence of SEQ ID NO: 7, preferably over the entire length of SEQ ID NO: 7, or a fragment, variant or homolog thereof (see FIG. 7).

According to a further aspect of the invention there is provided a polypeptide comprising the amino acid sequence of SEQ ID NO: 7.

According to a further aspect of the invention there is provided a polynucleotide comprising a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 7, preferably over the entire length of SEQ ID NO: 7, or a nucleotide sequence fully complementary to said polynucleotide.

According to a further aspect of the invention there is provided a polynucleotide which hybridises to a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO:7, preferably over the entire length of SEQ ID NO: 7.

According to a further aspect of the invention there is provided a polynucleotide comprising a nucleotide sequence encoding the polypeptide of SEQ ID NO: 7 or obtainable by screening an appropriate library under stringent hybridisation conditions with a probe.

According to a further aspect of the invention there is provided a polynucleotide comprising a nucleotide sequence which has at least 50%, preferably at least 70% homology or identity (preferably at least 80%, 85% 90%, 95% or 99% homology or identity) to the sequence of SEQ ID NO: 8, preferably over the entire length of SEQ ID NO: 8, or a nucleotide sequence fully complementary to said polynucleotide (see FIG. 8).

According to a further aspect of the invention there is provided a polynucleotide having the sequence of SEQ ID NO: 8.

According to a further aspect of the invention there is provided a polynucleotide comprising a nucleotide sequence which has at least 50%, preferably at least 70% homology or identity (preferably at least 80%, 85% 90%, 95% or 99% homology or identity) to the sequence of SEQ ID NO: 9, preferably over the entire length of SEQ ID NO: 9, or a nucleotide sequence fully complementary to said polynucleotide (see FIG. 9).

According to a further aspect of the invention there is provided a polynucleotide having the sequence of SEQ ID NO: 9.

According to a further aspect of the invention there is provided a polynucleotide comprising a nucleotide sequence which has at least 50%, preferably at least 70% homology or identity (preferably at least 80%, 85% 90%, 95% or 99% homology or identity) to the sequence of SEQ ID NO: 10, preferably over the entire length of SEQ ID NO: 10, or a nucleotide sequence fully complementary to said polynucleotide (see FIG. 10).

According to a further aspect of the invention there is provided a polynucleotide having the sequence of SEQ ID NO: 10.

According to a further aspect of the invention there is provided a polypeptide comprising an amino acid sequence which has at least 70% identity (preferably at least 80%, preferably at least 85%, preferably at least 90%, preferably at least 95% or at least 99% identity) to the amino acid sequence of SEQ ID NO: 11, preferably over the entire length of SEQ ID NO: 11, or a fragment, variant or homolog thereof (see FIG. 11).

According to a further aspect of the invention there is provided a polypeptide comprising the amino acid sequence of SEQ ID NO: 11.

According to a further aspect of the invention there is provided a polynucleotide comprising a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 11, preferably over the entire length of SEQ ID NO: 11, or a nucleotide sequence fully complementary to said polynucleotide.

According to a further aspect of the invention there is provided a polynucleotide which hybridises to a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 11, preferably over the entire length of SEQ ID NO: 11.

According to a further aspect of the invention there is provided a polynucleotide comprising a nucleotide sequence encoding the polypeptide of SEQ ID NO: 11 or obtainable by screening an appropriate library under stringent hybridisation conditions with a probe.

According to a further aspect of the invention there is provided a polypeptide comprising an amino acid sequence which has at least 70% identity (preferably at least 80%, preferably at least 85%, preferably at least 90%, preferably at least 95% or at least 99% identity) to the amino acid sequence of SEQ ID NO: 12, preferably over the entire length of SEQ ID NO: 12, or a fragment, variant or homolog thereof (see FIG. 12).

According to a further aspect of the invention there is provided a polypeptide comprising the amino acid sequence of SEQ ID NO: 12.

According to a further aspect of the invention there is provided a polynucleotide comprising a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 12, preferably over the entire length of SEQ ID NO: 12, or a nucleotide sequence fully complementary to said polynucleotide.

According to a further aspect of the invention there is provided a polynucleotide which hybridises to a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 12, preferably over the entire length of SEQ ID NO: 12.

According to a further aspect of the invention there is provided a polynucleotide comprising a nucleotide sequence encoding the polypeptide of SEQ ID NO: 12 or obtainable by screening an appropriate library under stringent hybridisation conditions with a probe.

According to a further aspect of the invention there is provided a polypeptide comprising an amino acid sequence which has at least 70% identity (preferably at least 80%, preferably at least 85%, preferably at least 90%, preferably at least 95% or at least 99% identity) to the amino acid sequence of SEQ ID NO: 13, preferably over the entire length of SEQ ID NO: 13, or a fragment, variant or homolog thereof (see FIG. 13).

According to a further aspect of the invention there is provided a polypeptide comprising the amino acid sequence of SEQ ID NO: 13.

According to a further aspect of the invention there is provided a polynucleotide comprising a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 13, preferably over the entire length of SEQ ID NO: 13, or a nucleotide sequence fully complementary to said polynucleotide.

According to a further aspect of the invention there is provided a polynucleotide which hybridises to a nucleotide sequence encoding a polypeptide which has at least 70% identity (preferably at least 80%, preferably at least 85%, preferably at least 90%, preferably at least 95% or at least 99% identity) to the amino acid sequence of SEQ ID NO: 13, preferably over the entire length of SEQ ID NO: 13.

According to a further aspect of the invention there is provided a polynucleotide comprising a nucleotide sequence encoding the polypeptide of SEQ ID NO: 13 or obtainable by screening an appropriate library under stringent hybridisation conditions with a probe.

According to a further aspect of the invention there is provided a vector comprising a polynucleotide as described above. Suitably in such a vector, the polynucleotide is operatively linked to an expression control sequence.

According to a further aspect of the invention there is provided a host cell stably transformed or transfected with a polynucleotide as described above in a manner allowing the expression in said host cell of a corresponding polypeptide.

According to a further aspect of the invention there is provided a method for producing a polypeptide as described above by growing a host cell as described above in a suitable nutrient medium and isolating the polypeptide.

According to a further aspect of the invention there is provided a method of identifying a compound that is a modulator of a polypeptide as described above or a polynucleotide as described above comprising the steps of:
a) determining the activity of said polypeptide in the presence and absence of said compound;
b) comparing the activities observed in step (a); and
c) identifying said compound as a modulator by the observed differences in the activity of said polypeptide in the presence and absence of said compound.

Suitably in such a method the activity is observed via modulation of the Notch signalling pathway.

According to a further aspect of the invention there is provided a modulator of a polypeptide as described above or a polynucleotide as described above identifiable using a method as described above.

According to a further aspect of the invention there is provided an antibody capable of specifically binding to a polypeptide as described above. Suitably the antibody may be a monoclonal antibody. According to a further aspect of the invention there is provided a hybridoma cell line producing such a monoclonal antibody. Preferably the antibody binds to the human protein homologue in preference to the corresponding mouse or other such homologue.

According to a further aspect of the invention there is provided a pharmaceutical composition comprising a polypeptide, polynucleotide or antibody as described above or a modulator as described above and a pharmaceutically acceptable diluent, carrier or excipient.

### BRIEF DESCRIPTION OF THE DRAWINGS

Various preferred features and embodiments of the present invention will now be described in more detail by way of non-limiting example and with reference to the accompanying drawings, in which:

FIG. 1A shows the fragment of human Notch1 intracellular domain (N1; SEQ ID NO: 1) corresponding to amino acids 1762-1829 as used in Example 1 (SEQ ID NO: 2).

FIG. 1B shows the coding sequence for the Notch 1 fragment as used in Example 1 (SEQ ID NO: 3).

FIG. 1C shows the amino acid sequence for the GAL4BD-N1 fusion as used in Example 1 (SEQ ID NO: 4), with the Notch 1 sequence shown underlined.

FIG. 2 shows the results of the GAL4BD (bait) construct specificity checks described in Example 2.

FIG. 3A shows the colonies identified in the yeast two-hybrid screen described in Example 3.

FIG. 3B shows the colonies identified in the yeast two hybrid screen described in Example 3, under 3-amino-1, 2, 4-triazole selection.

FIG. 4 shows the results of the prey (GAL4AD) construct specificity checks described in Example 4.

FIG. 5A shows the nucleotide sequence of the cDNA insert identified in Example 5 (SEQ ID NO: 5).

FIG. 5B shows the amino acid sequence coded by the cDNA insert identified in Example 5 (SEQ ID NO: 6).

FIG. 6 shows the nucleotide and amino acid sequences of FIGS. 5A (SEQ ID NO: 4 and SEQ ID NO: 5) and 5B (SEQ ID NO: 6) in alignment.

FIG. 7 shows the novel amino acid sequence coded by plasmid 8-7 as described in Example 5 (SEQ ID NO: 7).

FIG. 8 shows a proposed coding sequence for a human gene designated C2PDZRGS (SEQ ID NO: 8).

FIG. 9 shows a proposed coding sequence for a human homologue of the mouse gene C2PA (SEQ ID NO: 9).

FIG. 10 shows a proposed coding sequence for a human homologue of the mouse gene PDZ-RGS3 (SEQ ID NO: 10).

FIG. 11 shows a predicted amino acid sequence coded by a human gene C2PDZRGS (SEQ ID NO: 11).

FIG. 12 shows a predicted amino acid sequence coded by a human homologue of the mouse gene C2PA (SEQ ID NO: 12).

FIG. 13 shows a predicted amino acid sequence coded by a human homologue of the mouse gene PDZ-RGS3 (SEQ ID NO: 13).

FIG. 14 shows a schematic alignment between the splicing variant 8-7 and the human C2PA gene. Regions of nucleotide identity are shown within vertical bars. Arrows shows the borders of the ORF.

FIG. 15A shows a schematic alignment between five C2PA splicing variants identified in the mouse. The dotted block identifies the region of nucleotide identity and emboldened numbers mark the borders of the homologous region with respect to the sequence deposited in GenBank.

FIG. 15B shows a schematic alignment between the human and the mouse C2PA. The bars mark the region of identity between the splicing variants showing that the ORF of the human C2PA is completely within the identity region.

FIG. 16 shows schematically the result of mapping various mouse and human sequences.

FIG. 17 shows the results of the search described in Example 6.

FIG. 18 shows a schematic representation of Notch/Ligand interaction.

FIG. 19 shows a schematic representation of the Notch signalling pathway.

FIG. 20 shows a schematic representation of Notch 1-4.

FIG. 21 shows a schematic representation of Notch ligands Jagged and Delta.

FIG. 22 shows aligned amino acid sequences of DSL domains from various Drosophila and mammalian Notch ligands (SEQ ID NO: 14-29).

FIGS. 23A-23C show amino acid sequences of human Delta-1 (23A; SEQ ID NO: 30), Delta-2 (23B; SEQ ID NO: 31) and Delta-3 (23C; SEQ ID NO: 32).

FIGS. 24A and 24B show amino acid sequences of human Jagged-1 (SEQ ID NO: 33) and Jagged-2 (SEQ ID NO: 34), respectively.

FIG. 25 shows schematic representations of fusion proteins which may be used according to the present invention.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is herein incorporated by reference.

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists. Suitably the modulator is an agonist of Notch signalling, and preferably an agonist of the Notch receptor (eg an agonist of the human Notch1, Notch2, Notch3 and/or Notch4 receptor).

As used herein, the expression “Notch signalling” is synonymous with the expression “the Notch signalling pathway” and refers to any one or more of the upstream or downstream events that result in, or from, (and including) activation of the Notch receptor.

The term “chemokine” as used herein includes proteins that regulate cell (especially leukocyte) migration and activation. They are typically secreted by activated leukocytes themselves, and also by stromal cells such as endothelial and epithelial cells, after inflammatory stimuli. Examples of chemokines include monocyte chemotactic protein (MCP)-3, MCP4, macrophage inflammatory protein (MIP)-1a, MIP-1, B, RANTES (regulated on activation, normal T cell expressed and secreted), SDF-1, Teck (thymus expressed chemokine), and MDC (macrophage derived chemokine). As used herein, chemokine also includes any molecule that can act as a chemotactic agent. A chemotactic agent may be a small chemical compound, natural or synthetic, that is a selective agonist of a chemokine receptor, for example, CXCR4, a heterotrimeric G protein-coupled receptor (GPCR) that is the receptor for the chemokine SDF-1.

Altered cell migration includes a measurable or observable effect on cell migration by an agent, when compared to cell migration in the absence of said agent. Such altered migration may be detected using any suitable assay, for example a transwell migration assay (see for example Jo et al, J Clin Invest, 2000 January; 105 (1): 101-11 using SDF1).

Altered sensitivity to a chemokine includes an effect of a chemokine on a population of cells that is measurably or observably different under one set of conditions (for example, in the absence of a modulator of Notch signalling) from the effect of the same chemokine on the same population of cells under a different set of conditions (for example, in the presence of a modulator of Notch signalling).

The active agent of the present invention may be an organic compound or other chemical. In one embodiment, a modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the modulator will be an amino acid sequence or a chemical derivative thereof, or a combination thereof. In another preferred embodiment, the modulator will be a nucleotide sequence—which may be a sense sequence or an anti-sense sequence. The modulator may also be an antibody.

The term “antibody” includes intact molecules as well as fragments thereof, such as Fab, F(ab′)₂, Fv and scFv which are capable of binding the epitopic determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;
(iv) Fv, defined as a genetically engineered fragment containing the variable genetically fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

Modulators may be synthetic compounds or natural isolated compounds.

In one form the agent for modulation of the Notch signalling pathway may be a protein for Notch signalling transduction.

By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the protein is a domain that allows activation of target genes of the Notch signalling pathway, or a polynucleotide sequence which codes therefor.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In the present invention Notch signalling preferably means specific signalling, meaning that the signalling results substantially or at least predominantly from the Notch signalling pathway, and preferably from Notch/Notch ligand interaction, rather than any other significant interfering or competing cause, such as cytokine signalling. Thus, in a preferred embodiment, Notch signalling excludes cytokine signalling.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (eg chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or be employing other mechanisms to transduce extracellular signals.

According to one aspect of the present invention the active agent may be Notch or a fragment thereof which retains the signalling transduction ability of Notch or an analogue of Notch which has the signalling transduction ability of Notch.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a protein which is for Notch signalling activation we mean a molecule which is capable of activating Notch, the Notch signalling pathway or any one or more of the components of the Notch signalling pathway.

In a particular embodiment, the active agent will be capable of inducing or increasing Notch or Notch ligand expression. Such a molecule may be a nucleic acid sequence capable of inducing or increasing Notch or Notch ligand expression.

In one embodiment, the active agent will be capable of upregulating expression of the endogenous genes encoding Notch or Notch ligands in target cells. In particular, the molecule may be an immunosuppressive cytokine capable of upregulating the expression of endogenous Notch or Notch ligands in target cells, or a polynucleotide which encodes such a cytokine. Immunosuppressive cytokines include IL-4, IL-10, IL-13, TGF-β and SLIP3 (FLT3) ligand.

Preferably, the active agent will be a polypeptide selected from Noggin, Chordin, Follistatin, Xnr3, fibroblast growth factors and derivatives, fragments, variants and homologues thereof, or a polynucleotide encoding any one or more of the above.

In another embodiment, the active agent may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hematopoietic stem cells.

The term “Notch ligand” as used herein means an agent capable of interacting with a Notch receptor to cause a biological effect. The term as used herein therefore includes naturally occurring protein ligands such as Delta and Serrate/Jagged as well as antibodies to the Notch receptor, peptidomimetics and small molecules which have corresponding biological effects to the natural ligands. Preferably the Notch ligand interacts with the Notch receptor by binding.

Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive. For example, human Jagged-2 has 40.6% identity and 58.7% similarity to Serrate.

In a preferred embodiment, an activator will be a constitutively active Notch receptor or Notch intracellular domain, or a polynucleotide encoding such a receptor or intracellular domain.

In an alternative embodiment, an activator of Notch signalling will act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include the polypeptides involved in the Ras/MAPK cascade catalysed by Deltex, polypeptides involved in the proteolytic cleavage of Notch such as Presenilin and polypeptides involved in the transcriptional regulation of Notch target genes, preferably in a constitutively active form.

By polypeptide for Notch signalling activation is also meant any polypeptides expressed as a result of Notch activation and any polypeptides involved in the expression of such polypeptides, or polynucleotides coding for such polypeptides.

Activation of Notch signalling may also be achieved by repressing inhibitors of the Notch signalling pathway. As such, polypeptides for Notch signalling activation will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the molecules will be capable of repressing polypeptides of the Toll-like receptor protein family, cytokines such as IL-12, IFN-γ, TNF-α, and growth factors such as the bone morphogenetic protein (BMP), BMP receptors and activins, derivatives, fragments, variants and homologues thereof.

By a protein which is for Notch signalling inhibition or a polynucleotide encoding such a protein, we mean a molecule which is capable of inhibiting Notch, the Notch signalling pathway or any one or more of the components of the Notch signalling pathway.

In a particular embodiment, the molecule will be capable of reducing or preventing Notch or Notch ligand expression. Such a molecule may be a nucleic acid sequence capable of reducing or preventing Notch or Notch ligand expression.

Preferably the nucleic acid sequence encodes a polypeptide selected from Toll-like receptor protein family, a cytokine such as IL-12, IFN-γ, TNF-α, or a growth factor such as a bone morphogenetic protein (BMP), a BMP receptor and activins. Preferably the agent is a polypeptide, or a polynucleotide encoding such a polypeptide, that decreases or interferes with the production of compounds that are capable of producing an increase in the expression of Notch ligand, such as Noggin, Chordin, Follistatin, Xnr3, fibroblast growth factors and derivatives, fragments, variants and homologues thereof.

Alternatively, the nucleic acid sequence may be an antisense construct derived from a sense nucleotide sequence encoding a polypeptide selected from a Notch ligand and a polypeptide capable of upregulating Notch ligand expression, such as Noggin, Chordin, Follistatin, Xnr3, fibroblast growth factors and derivatives, fragments, variants and homologues thereof.

In another preferred embodiment a modulator of Notch signalling may be a molecule which is capable of modulating Notch-Notch ligand interactions. A molecule may be considered to modulate Notch-Notch ligand interactions if it is capable of inhibiting the interaction of Notch with its ligands, preferably to an extent sufficient to provide therapeutic efficacy.

The molecule may also be a polypeptide, or a polynucleotide encoding such a polypeptide, selected from a Toll-like receptor, a cytokine such as IL-12, IFN-γ, TNF-α, or a growth factor such as a BMP, a BMP receptor and activins. Preferably the polypeptide decreases or interferes with the production of an agent that is capable of producing an increase in the expression of Notch ligand, such as Noggin, Chordin, Follistatin, Xnr3, fibroblast growth factors and derivatives, fragments, variants, homologues and analogues thereof.

Preferably when the inhibitor is a receptor or a nucleic acid sequence encoding a receptor, the receptor is activated. Thus, for example, when the agent is a nucleic acid sequence, the receptor is preferably constitutively active when expressed.

Inhibitors of Notch signalling also include downstream inhibitors of the Notch signalling pathway, compounds that prevent expression of Notch target genes or induce expression of genes repressed by the Notch signalling pathway. Examples of such proteins include Dsh and Numb and dominant negative versions of Notch IC and Deltex. Proteins for Notch signalling inhibition will also include variants of the wild-type components of the Notch signalling pathway which have been modified in such a way that their presence blocks rather than transduces the signalling pathway. An example of such a compound would be a Notch receptor which has been modified such that proteolytic cleavage of its intracellular domain is no longer possible.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

Techniques for drug screening may be based on the method described in Geysen, European Patent No. 0138855, published on Sep. 13, 1984. In summary, large numbers of different small peptide candidate modulators or targeting molecules are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators and targeting molecules.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to components of the Notch signalling pathway. These include the use of phage display systems for expressing signalling proteins, and using a culture of transfected E. coli or other microorganism to produce the proteins for binding studies of potential binding compounds (see, for example, G. Cesarini, FEBS Letters, 307 (1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Chemokine Signalling

As further described, for example, in U.S. Pat. No. 6,506,777 it is well-known that in many inflammatory diseases, infiltration of leukocytes such as macrophages, neutrophils, eosinophils and lymphocytes into the inflammatory site is observed. It is thought that a physiologically active substance called chemotactic factor plays a role in the tissue accumulation of leukocytes. In particular, it has been reported that chemokines known as chemotactic cytokines induce not only infiltration of leukocytes, but also degranulation of leukocytes, production of active oxygen and adhesion reaction, and play central roles in the chemotaxis and activation of leukocytes (e.g., The New England Journal of Medicine, Vol. 338, pp. 436-445, 1998).

Chemokines are classified into 4 subgroups, i.e., C chemokines, CC chemokines, CXC chemokines and CXXXC chemokines, depending on the characteristics of the amino acid sequences (e.g., Blood, Vol. 90, pp. 909-928, 1997).

It is known that lymphotactins, to which C chemokines belong, have chemotactic activities to T lymphocytes, while CC chemokines induce chemotactic activities to leukocytes other than neutrophils, such as monocytes, lymphocytes, eosinophils, basophils and NK cells. CXC chemokines have chemotactic activities mainly to neutrophils and CXXC chemokines have chemotactic activities mainly to NK cells (e.g., The New England Journal of Medicine, Vol. 338, pp. 436-445, 1998). Such a cell specificity suggests that among these subgroups of chemokines, CC chemokines play important roles in the diseases including allergic diseases such as bronchial asthma and atopic dermatitis, chronic rheumatoid arthritis, sarcoidosis, pulmonary fibrosis, bacterial pneumonia, nephritis, atherosclerosis, ulcerative colitis, psoriasis, viral meningitis and AIDS. For example, in mice in which MIP-1alpha, one of the CC chemokines, is knocked out, the pneumonia induced by infection with influenza virus is reduced (Science, Vol. 269, pp. 1583-1585, 1995). In addition, it is known that in mice in which CCR1, one of the receptors of CC chemokines, is knocked out, the response by helper T cells type 2 which give important contribute to onset of atopic diseases is reduced (The Journal of Experimental Medicine, Vol. 185, pp. 1959-1968, 1997), and that in mice in which CCR2 is knocked out, delayed hypersensitivity reaction and response by helper T cells type 1 is reduced (The Journal of Clinical Investigation, Vol. 100, pp. 2552-2561, 1997). Further, it is known that in mice in which eotaxin, one of CC chemokines, is knocked out, tissue accumulation of eosinophils, which play important roles as effector cells in allergic diseases, occurs (The Journal of Experimental Medicine, Vol. 185, pp. 785-790, 1997). Further, it has been reported that CCR5 and CCR3, which are CC chemokine receptors, are cofactors in infection of AIDS virus, and that RANTES, MIP-1 alpha and MIP-1beta, which are CC chemokines prevent infection of AIDS virus (e.g., Annual Review of Immunology, Vol. 15, pp. 675-705, 1997).

Any of the cell types mentioned herein, including but not limited to macrophages, neutrophils, eosinophils, lymphocytes, neutrophils, monocytes, basophils and NK cells, can be a “target cell” for purposes of the invention.

The chemokines bind specific cell-surface receptors belonging to the family of G-protein-coupled seven-transmembrane-domain proteins (reviewed in Horuk, Trends Pharm. Sci., 15, 159-165 (1994)) which are termed “chemokine receptors.” On binding their cognate ligands, chemokine receptors transduce an intracellular signal though the associated trimeric G protein, resulting in a rapid increase in intracellular calcium concentration. There are at least sixteen human chemokine receptors that bind or respond to beta.-chemokines with the following characteristic pattern: CCR-1 (or “CKR-1” or “CC-CKR-1”) [MIP-1 alpha, MIP-1beta, MCP-3, RANTES] (Ben-Barruch, et al., J. Biol. Chem., 270, 22123-22128 (1995); eote, et al, Cell, 72, 415-425 (1993)); CCR-2A and CCR-2B (or “CKR-2A”/“CCKR-2A” or “CC-CKR-2A”/“CC-CKR-2A”) [MCP-1, MCP-3, MCP-4]; CCR-3 (or “CKR-3” or “CC-CKR-3”) [eotaxin, RANTES, MCP-3] (Combadiere, et al., J. Biol. Chem., 270, 16491-16494 (1995); CCR-4 (or “CKR-4” or “CC-CKR-4”) [MIP-1 alpha, RANTES, MCP-1] (Power, et al., J. Biol. Chem., 270, 19495-19500 (1995)); CCR-5 (or “CKR-5” or “CC-CKR-5”) [MIP-1alpha, RANTES, MIP-1beta] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The beta.-chemokines include eotaxin, MIP (“macrophage inflammatory protein”), MCP (“monocyte chemoattractant protein”) and RANTES (“regulation-upon-activation, normal T expressed and secreted”).

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. A review of the role of chemokines in allergic inflammation is provided by Kita, H., et al., J. Exp. Med. 183, 2421-2426 (1996). Accordingly, agents which modulate chemokine receptors are useful in such disorders and diseases. Compounds which modulate chemokine receptors are especially useful in the treatment and prevention of atopic conditions including allergic rhinitis, dermatitis, conjunctivitis, and particularly bronchial asthma.

For example, the natural ligand for CXCR4 is stromal cell-derived factor (SDF1). SDF1 alpha and 1 beta are small cytokines belonging to the CXC subfamily. The SDF1 alpha and SDF1 beta genes encode proteins of 89 and 93 amino acids, respectively (Shirozu et al., Genomics 28:495-500 (1995), see, also Genbank accession numbers L36033 and L36034).

As described, for example, in U.S. Pat. No. 6,395,497, chemokine receptors function in the migration of leukocytes throughout the body, particularly to inflammatory sites. Inflammatory cell emigration from the vasculature is regulated by a three-step process involving interactions of leukocyte and endothelial cell adhesion proteins and cell specific chemoattractants and activating factors (Springer, T. A., Cell, 76:301-314 (1994); Butcher, E. C., Cell, 67:1033-1036 (1991); Butcher, E. C. and Picker, L. J., Science (Wash. D.C.), 272:60-66 (1996)). These are: (a) a low affinity interaction between leukocyte selectins and endothelial cell carbohydrates; (b) a high-affinity interaction between leukocyte chemoattractant receptors and chemoattractant/activating factors; and (c) a tight-binding between leukocyte integrins and endothelial cell adhesion proteins of the immunoglobulin superfamily. Different leukocyte subsets express different repertoires of selecting, chemoattractant receptors and integrins. Additionally, inflammation alters the expression of endothelial adhesion proteins and the expression of chemoattractant and leukocyte activating factors. As a consequence, there is a great deal of diversity for regulating the selectivity of leukocyte recruitment to extravascular sites. The second step is crucial in that the activation of the leukocyte chemoattractant receptors is thought to cause the transition from the selectin-mediated cell rolling to the integrin-mediated tight binding. This results in the leukocyte being ready to transmigrate to perivascular sites. The chemoattractant/chemoattractant receptor interaction is also crucial for transendothelial migration and localization within a tissue (Campbell, J. J., et al., J. Cell Biol., 134:255-266 (1996); Carr, M. W., et al., Immunity, 4:179-187 (1996)). This migration is directed by a concentration gradient of chemoattractant leading towards the inflammatory focus.

Mammalian chemokine receptors thus provide a target for interfering with or promoting eosinophil and/or lymphocyte function in a mammal, such as a human. Compounds which inhibit or promote chemokine receptor function, are particularly useful for modulating eosinophil and/or lymphocyte function for therapeutic purposes. Accordingly, the present invention is useful in the prevention and/or treatment of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, a modulator of Notch signalling which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited. For example, eosinophilic infiltration to inflammatory sites (e.g., in asthma) can be inhibited according to the present method.

Similarly, a modulator of Notch signalling which promotes one or more functions of a mammalian chemokine receptor (e.g., a human chemokine) is administered to stimulate (induce or enhance) an inflammatory response, such as leukocyte emigration, chemotaxis, exocytosis (e.g., of enzymes, histamine) or inflammatory mediator release, resulting in the beneficial stimulation of inflammatory processes. For example, eosinophils can be recruited to combat parasitic infections.

Diseases and conditions associated with inflammation and infection can be treated using the method of the present invention. In a preferred embodiment, the disease or condition is one in which the actions of eosinophils and/or lymphocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderrna; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with promoters of chemokine receptor function, include, but are not limited to: immunosuppression, such as that in individuals with immunodeficiency syndromes such as AIDS, individuals undergoing radiation therapy, chemotherapy, therapy for autoimmune disease or other drug therapy (e.g., corticosteroid therapy), which causes immunosuppression; immunosuppression due congenital deficiency in receptor function or other causes; and infectious diseases, such as parasitic diseases, including, but not limited to helminth infections, such as nematodes (round worms); (Trichuriasis, Enterobiasis, Ascariasis, Hookworm, Strongyloidiasis, Trichinosis, filariasis); trematodes (flukes) (Schistosomiasis, Clonorchiasis), cestodes (tape worms) (Echinococcosis, Taeniasis saginata, Cysticercosis); visceral worms, visceral larva migrans (e.g., Toxocara), eosinophilic gastroenteritis (e.g., Anisaki spp., Phocanema ssp.), cutaneous larva migrans (Ancylostona braziliense, Ancylostoma caninum).

In one embodiment, the modulator of Notch signalling is an antagonist of chemokine receptor function. Accordingly, processes or cellular responses mediated by the binding of a chemokine to a receptor can be inhibited (reduced or prevented, in whole or in part), including leukocyte migration, integrin activation, transient increases in the concentration of intracellular free calcium [Ca²⁺]₁, and/or granule release of proinflammatory mediators.

The invention further relates to a method of treatment, including prophylactic and therapeutic treatments, of a disease associated with aberrant leukocyte recruitment and/or activation or mediated by chemokines or chemokine receptor function, including chronic inflammatory disorders characterized by the presence of RANTES, MIP-1 alpha, MCP-2, MCP-3 and/or MCP-4 responsive T cells, monocytes and/or eosinophils, including but not limited to diseases such as arthritis (e.g., rheumatoid arthritis), atherosclerosis, arteriosclerosis, ischemia/reperfusion injury, diabetes mellitus (e.g., type 1 diabetes mellitus), psoriasis, multiple sclerosis, inflammatory bowel diseases such as ulcerative colitis and Crohn's disease, rejection of transplanted organs and tissues (i.e., acute allograft rejection, chronic allograft rejection), graft versus host disease, as well as allergies and asthma. Other diseases associated with aberrant leukocyte recruitment and/or activation which can be treated (including prophylactic treatments) with the methods disclosed herein are inflammatory diseases associated with Human Immunodeficiency Virus (HIV) infection, e.g., AIDS associated encephalitis, AIDS related maculopapular skin eruption, AIDS related interstitial pneumonia, AIDS related enteropathy, AIDS related periportal hepatic inflammation and AIDS related glomerulo nephritis. The method comprises administering to the subject in need of treatment an effective amount of a compound (i.e., one or more compounds) which inhibits chemokine receptor function, inhibits the binding of a chemokine to leukocytes and/or other cell types, and/or which inhibits leukocyte migration to, and/or activation at, sites of inflammation.

The invention further relates to methods of antagonizing a chemokine receptor comprising administering to the mammal a modulator of Notch signalling as described herein.

For example, chemokine-mediated chemotaxis and/or activation of pro-inflammatory cells bearing receptors for chemokines can be inhibited. As used herein, “pro-inflammatory cells” includes but is not limited to leukocytes, since chemokine receptors can be expressed on other cell types, such as neurons and epithelial cells.

As used herein, the term “inhibition of chemokine signalling” means inhibition (decrease or prevention) of at least one function characteristic of chemokine signalling such as binding activity (e.g., ligand binding, promoter binding, antibody binding), signaling activity (e.g., activation of a mammalian G protein, induction of rapid and transient increase in the concentration of cytosolic free calcium [Ca²⁺]₁), and/or cellular response function (e.g., stimulation of chemotaxis, exocytosis or inflammatory mediator release by leukocytes).

As used herein, the term “promotion of chemokine signalling” means promotion (enhancement or increase) of at least one function characteristic of chemokine signalling such as a binding activity (e.g., ligand, inhibitor and/or promoter binding), signaling activity (e.g., activation of a mammalian G protein, induction of rapid and transient increase in the concentration of cytosolic free calcium [Ca 2+]₁), and/or a cellular response function (e.g., stimulation of chemotaxis, exocytosis or inflammatory mediator release by leukocytes).

Thus, the present invention provides a method of inhibiting leukocyte trafficking in a mammal (e.g., a human patient), comprising administering to the mammal an effective amount of a modulator of Notch signalling.

Ephrin Signalling

The present invention further provides a method of modifying ephrin signalling by administering a modulator of the Notch signalling pathway.

As disclosed, for example, in WO02079382 (Harvard College) ligands in the ephrin-B family are cell surface anchored by a transmembrane domain, and signal through their Eph receptors by direct cell-cell contact (e.g. see Davis et al, 1994; Ligands for EPH-related receptor tyrosine kinases that require membrane attachment or clustering for activity. Science 266, 816-819). This contact-mediated mechanism provides the potential for bi-directional signalling, with a forward signal through the tyrosine kinase receptor, and a reverse signal through the ligand. Reverse signaling has been demonstrated biochemically by studies showing B ephrins become phosphorylated upon treatment of cells with soluble EphB-Fc receptor fusion protein (Holland et al (1996) Bidirectional signalling through the EPH-family receptor Nuk and its transmembrane ligand. Nature 383, 722-725). In the context of whole organisms or tissues, genetic and embryological studies have supported important roles for B ephrin reverse signaling in developmental processes, including axon pathway selection, blood vessel formation, and rhombomere compartmentation. The term “ephrin signalling” as used herein thus includes signalling by ephrin receptors or their ligands or both.

Proteins, Polypeptides and Polynucleotides

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required target activity or ability to modulate Notch signalling. Amino acid substitutions may include the use of non-naturally occurring analogues.

The protein used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the target or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Sym- | | | |
| bol | 3-letter | Meaning | Codons |

| | | | |
|---|---|---|---|
| A | Ala | Alanine | GCT,GCC,GCA,GCG |
| B | Asp,Asn | Aspartic, | GAT,GAC,AAT,AAC |
| | | Asparagine | |
| C | Cys | Cysteine | TGT,TGC |
| D | Asp | Aspartic | GAT,GAC |
| E | Glu | Glutamic | GAA,GAG |
| F | Phe | Phenylalanine | TTT,TTC |
| G | Gly | Glycine | GGT,GGC,GGA,GGG |
| H | His | Histidine | CAT,CAC |
| I | Ile | Isoleucine | ATT,ATC,ATA |
| K | Lys | Lysine | AAA,AAG |
| L | Leu | Leucine | TTG,TTA,CTT,CTC,CTA,CTG |
| M | Met | Methionine | ATG |
| N | Asn | Asparagine | AAT,AAC |
| P | Pro | Proline | CCT,CCC,CCA,CCG |
| Q | Gln | Glutamine | CAA,CAG |
| R | Arg | Arginine | CGT,CGC,CGA,CGG,AGA,AGG |
| S | Ser | Serine | TCT,TCC,TCA,TCG,AGT,AGC |
| T | Thr | Threonine | ACT,ACC,ACA,ACG |
| V | Val | Valine | GTT,GTC,GTA,GTG |
| W | Trp | Tryptophan | TGG |
| X | Xxx | Unknown | |
| Y | Tyr | Tyrosine | TAT,TAC |
| Z | Glu,Gln | Glutamic, | GAA,GAG,CAA,CAG |
| | | Glutamine | |
| * | End | Terminator | TAA,TAG,TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function. A peptide useful in the invention will at least have a target or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, between about 8 and about 745 amino acids in length, preferably about 8 to about 300, more preferably about 8 to about 200 amino acids, and even more preferably about 10 to about 50 or 100 amino acids in length. “Peptide” refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefore gene expression. The redundancy of the genetic code means that several different codons may encode the same amino acid. For example, leucine, arginine and serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

Where the active agent is a nucleotide sequences it may suitably be codon optimised for expression in mammalian cells. In a preferred embodiment, such sequences are optimised in their entirety.

“Polynucleotide” refers to a polymeric form of nucleotides of at least 10 bases in length and up to 10,000 bases or more, either ribonucleotides or deoxyribonucleotides or a modified form of either type of nucleotide. The term includes single and double stranded forms of DNA and also derivatised versions such as protein nucleic acid (PNA).

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Nucleic acid encoding the second region may likewise be provided in a similar vector system.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data are available or where it is desired to express a nucleic acid homologous or otherwise related to a known nucleic acid, exemplary nucleic acids can be characterised as those nucleotide sequences which hybridise to the nucleic acid sequences known in the art.

It will be understood by a skilled person that numerous different nucleotide sequences can encode the same protein used in the present invention as a result of the degeneracy of the genetic code. In addition, it is to be understood that skilled persons may, using routine techniques, make nucleotide substitutions that do not affect the protein encoded by the nucleotide sequence of the present invention to reflect the codon usage of any particular host organism in which the target protein or protein for Notch signalling modulation of the present invention is to be expressed.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a target protein or protein for T cell signalling modulation.

As indicated above, with respect to sequence homology, preferably there is at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the target protein or protein for T cell signalling modulation encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transvection, microinjection, cationic lipid-mediated transfection, electroporation, transduction, scrape loading, ballistic introduction and infection. In will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

A detailed description of the Notch signalling pathway and conditions affected by it may be found in our WO98/20142, WO00/36089 and WO0135990.

Polypeptides and Polynucleotides for Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as disulphide-linked heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas). Schematic representations of Notch and the Notch intracellular domain are shown in the accompanying Figures.

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc 10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc 10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the cdc 10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

S3 processing occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes) (Irvine).

Signal transduction from the Notch receptor can occur via two different pathways (see Figures). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF 1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

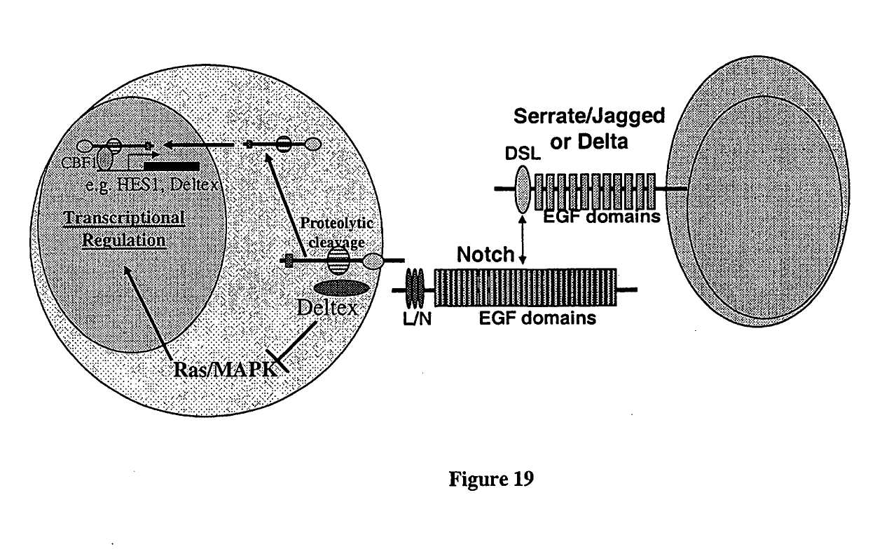

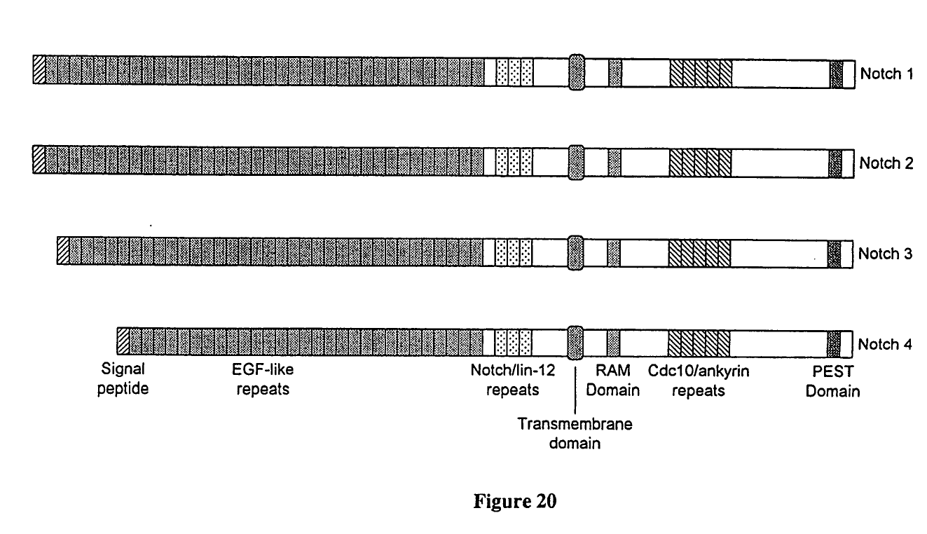

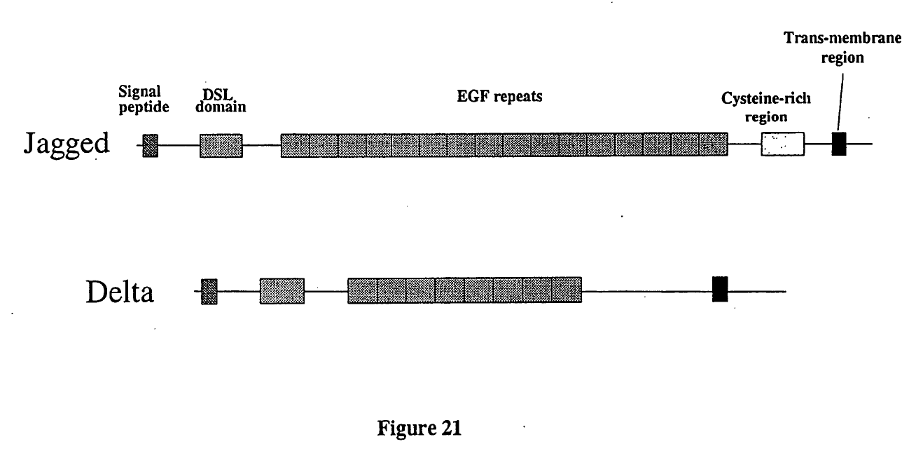

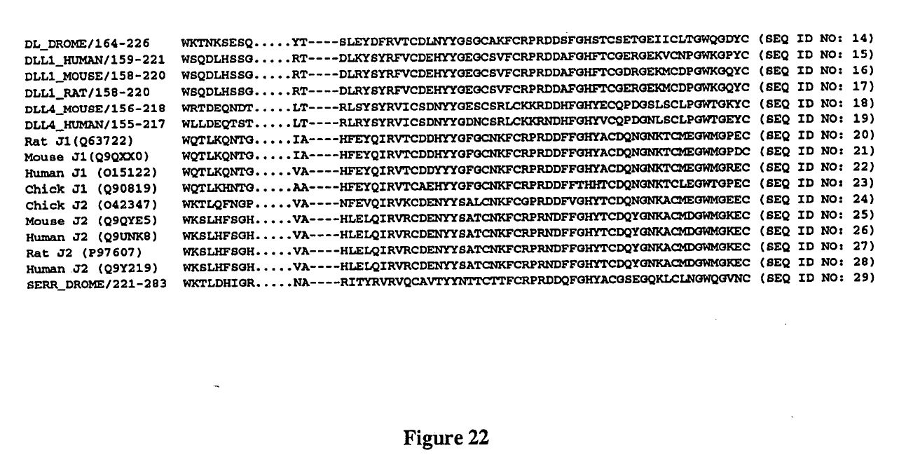

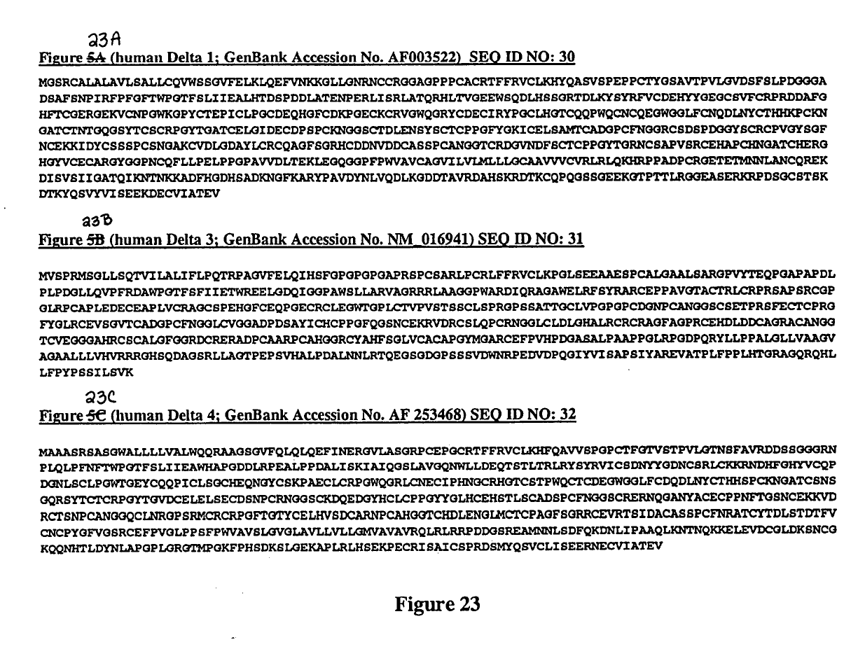

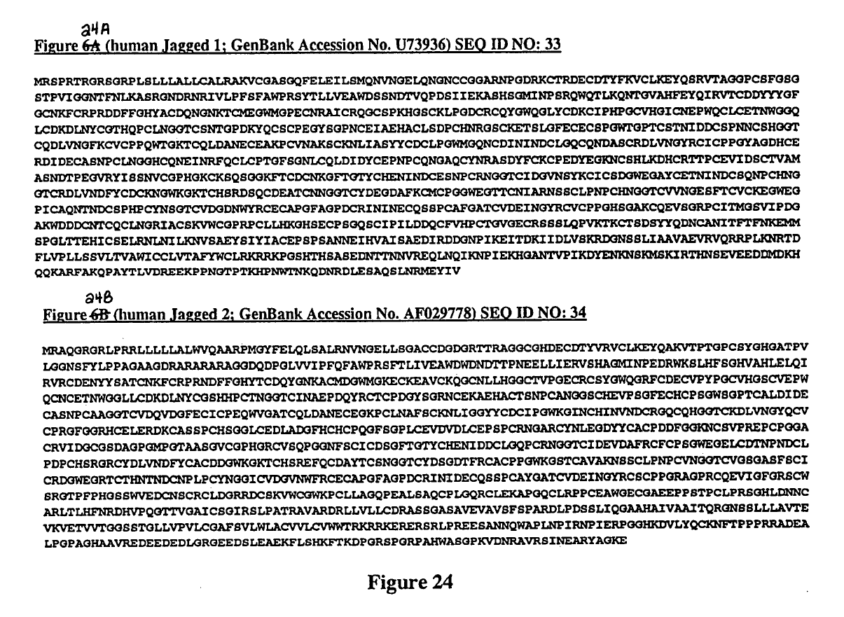

Thus, signal transduction from the Notch receptor can occur via two different pathways both of which are illustrated in FIG. 19. Target genes of the Notch signalling pathway include Deltex, genes of the Hes family (Hes-1 in particular), Enhancer of Split [E(spl)] complex genes, IL-10, CD-23, CD-4 and D11-1.

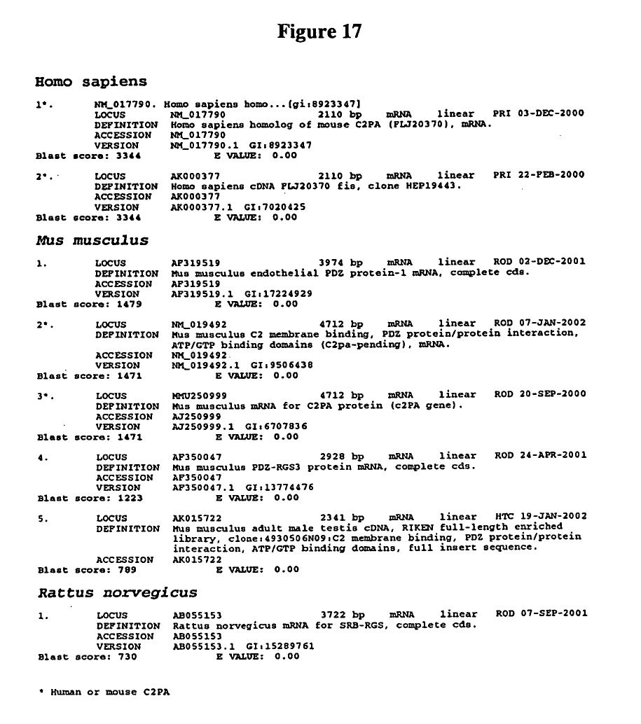

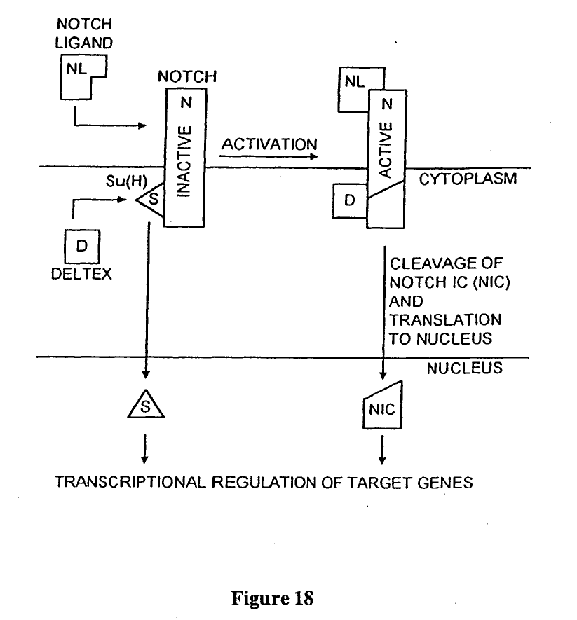

Deltex, an intracellular docking protein, replaces Su(H) as it leaves its site of interaction with the intracellular tail of Notch, as shown in FIG. 17. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

IL-10 (interleukin-10) is a factor produced by Th2 helper T-cells. It is a co-regulator of mast cell growth and shows extensive homology with the Epstein-Barr bcrfi gene. Although it is not known to be a direct downstream target of the Notch signalling pathway, its expression has been found to be strongly upregulated coincident with Notch activation. The mRNA sequence of IL-10 may be found in GenBank ref. No. G11041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. Although it is not thought to be a direct downstream target of the Notch signalling pathway, its expression has been found to be strongly upregulated coincident with Notch activation. The sequence for CD-23 may be found in GenBank ref. No. GI1783344.

Dlx-1 (distalless-1) (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Polypeptides and Polynucleotides for Notch Signalling Activation

Examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive. For example, human Jagged-2 has 40.6% identity and 58.7% similarity to Serrate.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequnce of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Other substances capable of activating the Notch signalling pathway include compounds capable of upregulating Notch ligand expression including polypeptides that bind to and reduce or neutralise the activity of bone morphogenetic proteins (BMPs). Binding of extracellular BMPs (Wilson and Hemmati-Brivanlou, Hemmati-Brivanlou and Melton) to their receptors leads to down-regulated Delta transcription due to the inhibition of the expression of transcription factors of the achaete/scute complex. This complex is believed to be directly involved in the regulation of Delta expression. Thus, any substance that inhibits BMP expression and/or inhibits the binding of BMPs to their receptors may be capable of producing an increase in the expression of Notch ligands such as Delta and/or Serrate. Particular examples of such inhibitors include Noggin (Valenzuela), Chordin (Sasai), Follistatin (lemura), Xnr3, and derivatives and variants thereof. Noggin and Chordin bind to BMPs thereby preventing activation of their signalling cascade which leads to decreased Delta transcription. Consequently, increasing Noggin and Chordin levels may lead to increase Notch ligand, in particular Delta, expression.

Furthermore, any substance that upregulates expression of transcription factors of the achaete/scute complex may also upregulate Notch ligand expression.

Other suitable substances that may be used to upregulate Notch ligand expression include transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao). Most preferably, the FGF is a member of the FGF family which acts by stimulating the upregulation of expression of a Serrate polypeptide on APCs. It has been shown that members of the FGF family can upregulate Serrate-1 gene expression in APCs.

Immunosuppressive cytokines may also be used to upregulate Notch ligand expression. Examples include members of the TGF-β family such as TGF-β-1 and TGF-β-2, and interleukins such as IL-4, IL-10, IL-4 and IL-13, and FLT3 ligand. The TGF-family can upregulate Notch, particularly Notch 1, expression; IL-10 can upregulate Serrate, particularly Serrate 1, expression; IL-10 can upregulate Notch, Delta and Serrate, particularly Notch 2, Notch 4, Delta 1 and Serrate 1, expression; and IL-10 can upregulate Serrate, particularly Serrate 1, expression.

The substance capable of upregulating expression of Notch or a Notch ligand may be selected from polypeptides and fragments thereof, linear peptides, cyclic peptides, including synthetic and natural compounds. The substances capable of upregulating expression of a Notch ligand may be derived from a biological material such as a component of extracellular matrix. Suitable extracellular matrix components are derived from immunologically privileged sites such as the eye. For example aqueous humour or components thereof may be used.

Polypeptide substances such as Noggin, FGFs and TGF-β, may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz) and other ADAMALYSIN gene family members.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO: 51):

Cys Xaa Xaa Xaa Xaa Xaa Xaa Xaa Xaa Cys Xaa Xaa
Xaa Cys Xaa Xaa Xaa Xaa Xaa Xaa Xaa Xaa Xaa Xaa
Xaa Cys Xaa Xaa Xaa Xaa Xaa Xaa Xaa Cys Xaa Xaa
Xaa Xaa Xaa Xaa Xaa Xaa Cys

Preferably the DSL domain may include most or all of the following consensus amino acid sequence (SEQ ID NO: 52):

Cys Xaa Xaa Xaa ARO ARO Xaa Xaa Xaa Cys Xaa Xaa
Xaa Cys BAS NOP BAS ACM ACM Xaa ARO NOP ARO Xaa
Xaa Cys Xaa Xaa Xaa NOP Xaa Xaa Xaa Cys Xaa Xaa
NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence (SEQ ID NO:53):

Cys Xaa Xaa Xaa Tyr Tyr Xaa Xaa Xaa Cys Xaa Xaa
Xaa Cys Arg Pro Arg Asx Asp Xaa Phe Gly His Xaa
Xaa Cys Xaa Xaa Xaa Gly Xaa Xaa Xaa Cys Xaa Xaa
Gly Trp Xaa Gly Xaa Xaa Cys

(wherein Xaa may be any amino acid and Asx is either aspartic acid or asparagine).

An alignment of DSL domains from Notch ligands from various sources is shown in the accompanying Figures.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain: 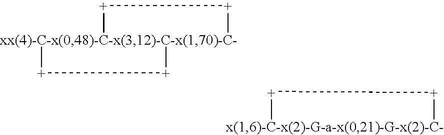
wherein:
‘C’: conserved cysteine involved in a disulfide bond.
‘G’: often conserved glycine
‘a’: often conserved aromatic amino acid
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X% identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

The term “heterologous amino acid sequence” or “heterologous nucleotide sequence” as used herein means a sequence which is not found in the native Notch ligand or its coding sequence.

Whether a substance can be used for activating Notch may be determined using suitable screening assays, for example, as described in our co-pending International Patent Application claiming priority from GB 0118153.6, and the examples herein.

Activation of Notch signalling may also be achieved by repressing inhibitors of the Notch signalling pathway. As such, polypeptides for Notch signalling activation will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the molecules will be capable of repressing polypeptides of the Toll-like receptor protein family and growth factors such as the bone morphogenetic protein (BMP), BMP receptors and activins, derivatives, fragments, variants and homologues thereof.

Polypeptides and Polynucleotides for Notch Signalling Inhibition

Substances that may be used to modulate Notch signalling by inhibiting Notch ligand expression include nucleic acid sequences encoding polypeptides that affect the expression of genes encoding Notch ligands. For instance, for Delta expression, binding of extracellular BMPs (bone morphogenetic proteins, Wilson and Hemmati-Brivanlou; Hemmati-Brivanlou and Melton) to their receptors leads to down-regulated Delta transcription due to the inhibition of the expression of transcription factors of the achaete/scute complex. This complex is believed to be directly involved in the regulation of Delta expression. Thus, any polypeptide that upregulates BMP expression and/or stimulates the binding of BMPs to their receptors may be capable of producing a decrease in the expression of Notch ligands such as Delta and/or Serrate. Examples may include nucleic acids encoding BMPs themselves. Furthermore, any substance that inhibits expression of transcription factors of the achaete/scute complex may also downregulate Notch ligand expression.

Members of the BMP family include BMP1 to BMP6, BMP7 also called OP1, OP2 (BMP8) and others. BMPs belong to the transforming growth factor beta (TGF-beta) superfamily, which includes, in addition to the TGF-betas, activins/inhibins (e.g., alpha-inhibin), mullerian inhibiting substance, and glial cell line-derived neurotrophic factor.

Other examples of polypeptides that inhibit the expression of Delta and/or Serrate include the Toll-like receptor (Medzhitov) or any other receptors linked to the innate immune system (for example CD14, complement receptors, scavenger receptors or defensin proteins), and other polypeptides that decrease or interfere with the production of Noggin (Valenzuela), Chordin (Sasai), Follistatin (Iemura), Xnr3, and derivatives and variants thereof. Noggin and Chordin bind to BMPs thereby preventing activation of their signalling cascade which leads to decreased Delta transcription. Consequently, reducing Noggin and Chordin levels may lead to decrease Notch ligand, in particular Delta, expression.

In more detail, in Drosophila, the Toll transmembrane receptor plays a central role in the signalling pathways that control amongst other things the innate nonspecific immune response. This Toll-mediated immune response reflects an ancestral conserved signalling system that has homologous components in a wide range of organisms. Human Toll homologues have been identified amongst the Toll-like receptor (TLR) genes and Toll/interleukin-1 receptor-like (TIL) genes and contain the characteristic Toll motifs: an extracellular leucine-rich repeat domain and a cytoplasmic interleukin-1 receptor-like region. The Toll-like receptor genes (including TIL genes) now include TLR4, TIL3, TIL4, and 4 other identified TLR genes.

Other suitable sequences that may be used to downregulate Notch ligand expression include those encoding immune costimulatory molecules (for example CD80, CD86, ICOS, SLAM) and other accessory molecules that are associated with immune potentiation (for example CD2, LFA-1).

Other suitable substances that may be used to downregulate Notch ligand expression include nucleic acids that inhibit the effect of transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao et al., 1995). Most preferably, the FGF is a member of the FGF family which acts by stimulating the upregulation of expression of a Serrate polypeptide on APCs. It has been shown that members of the FGF family can upregulate Serrate-1 gene expression in APCs.

Suitable nucleic acid sequences may include anti-sense constructs, for example nucleic acid sequences encoding antisense Notch ligand constructs as well as antisense constructs designed to reduce or inhibit the expression of upregulators of Notch ligand expression (see above). The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression, or Delta but not Serrate expression in APCs or T cells. Alternatively, the nucleic acid sequence for use in the present invention is capable of inhibiting Delta expression in T cells such as CD4+ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate expression in APCs and Delta expression in T cells.

Preferred suitable substances that may be used to downregulate Notch ligand expression include growth factors and cytokines. More preferably soluble protein growth factors may be used to inhibit Notch or Notch ligand expression. For instance, Notch ligand expression may be reduced or inhibited by the addition of BMPs or activins (a member of the TGF-β superfamily). In addition, T cells, APCs or tumour cells could be cultured in the presence of inflammatory type cytokines including IL-12, IFN-γ, IL-18, TNF-α, either alone or in combination with BMPs.

Molecules for inhibition of Notch signalling will also include polypeptides, or polynucleotides which encode therefore, capable of modifying Notch-protein expression or presentation on the cell membrane or signalling pathways. Molecules that reduce or interfere with its presentation as a fully functional cell membrane protein may include MMP inhibitors such as hydroxymate-based inhibitors.

Other substances which may be used to reduce interaction between Notch and Notch ligands are exogenous Notch or Notch ligands or functional derivatives thereof. Such Notch ligand derivatives would preferably have the DSL domain at the N-terminus and up to about 14 or more, for example between about 3 to 8 EGF-like repeats on the extracellular surface. A peptide corresponding to the Delta/Serrate/LAG-2 domain of hjaggedl and supernatants from COS cells expressing a soluble form of the extracellular portion of hjaggedl was found to mimic the effect of Jagged1 in inhibiting Notch1 (Li).

Other Notch signalling pathway antagonists include antibodies which inhibit interactions between components of the Notch signalling pathway, e.g. antibodies to Notch ligands.

Whether a substance can be used for modulating Notch-Notch ligand expression may be determined using suitable screening assays.

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of D11-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Antigens and Allergens

An antigen suitable for use in the present invention may be any substance that can be recognised by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFN-γ but low IL-4 production, whereas in humoral immunity (TH2) the pattern is low IL-2 and IFN-γ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens. Where primed the APCs and/or T cells of the present invention are to be used in tissue transplantation procedures, antigens may be obtained from the tissue donor.

The antigen or allergen moiety may be, for example, a synthetic MHC-peptide complex i.e. a fragment of the MHC molecule bearing the antigen groove bearing an element of the antigen. Such complexes have been described in Altman et al., 1996.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of fetal calf serum. Cytokines, if present, are typically added at up to 1000 U/ml. Optimum concentrations may be determined by titration. One or more substances capable of modulating presenilin and, optionally, one or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of a Notch signalling are now ready for use.

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s) capable of modulating presenilin to that used with the APC, then the T cell will not be brought into contact with the different substance(s) used in the APC. Alternatively, the T cell may be incubated with a first substance (or set of substances) to modulate presenilin or presenilin-dependent gamma-secretase and, optionally, Notch signalling, washed, resuspended and then incubated with the primed APC in the absence of both the substance(s) used to modulate the APC and the substance(s) used to modulate the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3 or 6 hours, in suitable culture medium at 37° C. Induction of immunotolerance may be determined by subsequently challenging T cells with antigen and measuring IL-2 production compared with control cells not exposed to APCs.

T cells or B cells which have been primed in this way may be used according to the invention to induce immunotolerance in other T cells or B cells.

Therapeutic Uses

Immunological Indications

In the preferred embodiment the therapeutic effect results from a protein for Notch signalling. A detailed description of the Notch signalling pathway and conditions affected by it may be found in our WO98/20142, WO00/36089 and PCT/GB00/04391.

Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple schlerosis, rheumatoid arthritis and diabetes. The present invention may also be used in organ transplantation or bone marrow transplantation.

As indicated above, the present invention is useful in treating immune disorders such as autoimmune diseases or graft rejection such as allograft rejection.

Autoimmune Disease

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, scleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy. The present invention is especially useful in relation to adenocarcinomas such as: small cell lung cancer, and cancer of the kidney, uterus, prostrate, bladder, ovary, colon and breast.

Transplant Rejection

The present invention may be used, for example, for the treatment of organ transplants (e.g. kidney, heart, lung, liver or pancreas transplants), tissue transplants (e.g. skin grafts) or cell transplants (e.g. bone marrow transplants or blood transfusions).

A brief overview of the most common types of organ and tissue transplants is set out below.

1. Kidney Transplantation

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

2. Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

3. Lung Transplantation

Lung transplantation is used (either by itself or in combination with heart transplantation) to treat diseases such as cystic fibrosis and acute damage to the lungs (e.g. caused by smoke inhalation). Lungs for use in transplants are normally recovered from brain-dead donors.

4. Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

5. Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in burn victims.

6. Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by liver transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

Cell Fate/Cancer Indications

The present invention is also useful in methods for altering the fate of a cell, tissue or organ type by altering Notch pathway function in the cell. Thus, the present application has application in the treatment of malignant and pre-neoplastic disorders. The present invention is especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sewat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

The present invention may also have application in the treatment of nervous system disorders. Nervous system disorders which may be treated according to the present invention include neurological lesions including traumatic lesions resulting from physical injuries; ischaemic lesions; malignant lesions; infectious lesions such as those caused by HIV, herpes zoster or herpes simplex virus, Lyme disease, tuberculosis or syphilis; degenerative lesions and diseases and demyelinated lesions.

The present invention may be used to treat, for example, diabetes (including diabetic neuropathy, Bell's palsy), systemic lupus erythematosus, sarcoidosis, multiple sclerosis, human immunodeficiency virus-associated myelopathy, transverse myelopathy or various etiologies, progressive multifocal leukoencephalopathy, central pontine myelinolysis, Parkinson's disease, Alzheimer's disease, Huntington's chorea, amyotrophic lateral sclerosis, cerebral infarction or ischemia, spinal cord infarction or ischemia, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

The present invention may further be useful in the promotion of tissue regeneration and repair. The present invention, therefore, may also be used to treat diseases associated with defective tissue repair and regeneration such as, for example, cirrhosis of the liver, hypertrophic scar formation and psoriasis. The invention may also be useful in the treatment of neutropenia or anemia and in techniques of organ regeneration and tissue engineering.

Pharmaceutical Compositions

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (Ed. A. R. Gennaro 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient.

In general, a therapeutically effective daily dose of the conjugate of an active agent according to the invention invention may for example range from 0.01 to 50 mg/kg body weight of the subject to be treated, preferably 0.1 to 20 mg/kg.

A skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

Various preferred features and embodiments of the present invention will now be described in more detail by way of non-limiting examples.

### EXAMPLES

Reagents

In the following examples, the reagents were as follows:
10×YNB: 33.5 g YNB minus amino acids (Sigma) was dissolved in 500 ml MQ-H₂O and filter sterilised.
Glucose solution: 400 g Glucose was dissolved in 1 L MQ-H₂O and filter sterilised.
10× WHAUL dropout solution: The following were dissolved in 1 L of MQ-H₂O, and the resulting solution filter sterilised; 300 mg L-Isoleucine, 1.5 g L-Valine, 200 mg L-Arginine, 300 mg L-Lysine, 200 mg L-Methionine, 500 mg L-Phenylalanine, 2 g L-Threonine, 300 mg L-Tyrosine, 1 g L-Glutamic Acid, 1 g L-Aspartic Acid and 400 mg L-Serine.
Selection media & plates: The following were mixed; 160 ml MQ-H₂O, 20 ml 10×YNB, 20 ml 10×-WHAUL and 4 g Agar if plates required. The solution was autoclaved and cooled to 55° C. The following were then added and plates poured or media cooled for use: 10 ml 40% Glucose solution, 2 ml L-Uracil (2.4 mg/ml), 5 ml L-Adenine (5 mg/ml)*, 2 ml L-Histidine (2.4 mg/ml)*, 2 ml L-Leucine (7.2 mg/ml)*, and 2 ml L-Tryptophan (4.8 mg/ml)*. The amino acids denoted by * were added as required.
YPAD: The following were mixed; 10 g Difco peptone, 5 g Yeast extract and 10 g Agar added if plates required. The solution was then autoclaved and cooled to 55° C. The following were added and plates poured or media cooled for use; 25 ml Glucose solution, 200 mg Adenine sulphate.
LiAc/TE: 1×TE, 100 mM LiAc
LiAC/TE/PEG: 1×TE, 100 mM LiAc, 40% PEG (PEG 3350, Sigma)
Z Buffer: The following were dissolved in 900 ml MQ-H₂O, and the volume adjusted to 1 L: 16.1 g Na₂HPO₄.7H₂O, 5.5 g Na₂HPO₄.H₂O, 0.75 g KCl and 0.246 g MgSO₄. 7H₂O. The solution was then autoclaved before use.
Z Buffer with X-gal: The following were mixed just before use: 9.8 ml Z buffer, 27 μl β-mercaptoethanol, 167 μl X-gal [20 mg/ml in DMF]
Lyticase miniprep solution: 6 ml 2M sorbitol, 1 ml 1M KPO₄ pH 7.4, 2 ml Lyticase (4 mg/ml), 1 ml MQ-H₂O.
Template solution: 50 μl lyticase PCR solution, 2 μl P mercaptoethanol, 950 μl water
Lyticase PCR solution: 200 U/ml Lyticase (Sigma L-2524) in 50 mM Tris-HCL pH7.5, 1 mM EDTA, 50% Glycerol

### Example 1

### Preparation of Bait Construct

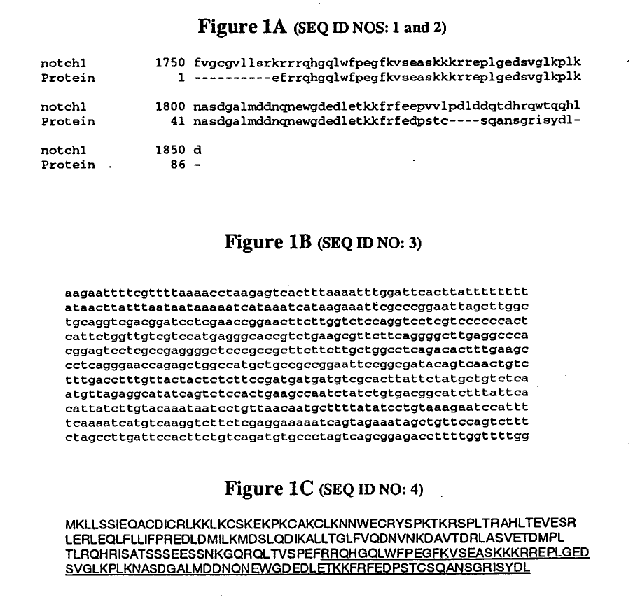

A fragment of hNotchl (AF308602 (DNA)/AAG33848 (protein)) intracellular domain (N1) (FIG. 1A; SEQ ID NO: 1) corresponding to amino acids 1762-1829 (FIG. 1A; SEQ ID NO: 2) which encodes the RAM23 to first predicted nuclear localisation sequence was generated by PCR using IMAGE clone 28967 as a template as follows:

Primers:
Forward (PH95)

(SEQ ID NO: 35)

TTT GAA TTC CGG CGG CAG CAT GGC CAG CTC
Reverse (PH108)

(SEQ ID NO: 36)

TTT GGA TCC TCG AAC CGG AAC TTC TTG GTC TCC

| | |
|---|---|
| Reaction Mix: | |
| Image clone 28967 DNA | 1 μl |
| 10× Buffer | 5 μl |
| PH95 (10 μM) | 2 μl |
| PH108 (10 μM) | 2 μl |
| dNTPs (10 μM) | 2 μl |
| Advantage 2 polymerase (Clontech) | 0.5 μl |
| Water | 35 μl |
| PCR cycle: | |
| 95° C. for 30 seconds | |
| 60° C. for 30 seconds | |
| 68° C. for 1 minute | |
| 25 cycles | |

The PCR reaction was cleaned using Qiaquick PCR clean up kit according to the manufacturer's instructions and the PCR fragment was then sub-cloned into Multi-cloning site 1 of pLOR32 (PBRIDGE with ampicillin resistance substituted with kanamycin resistance) using EcoRI and BamHI (highlighted in bold type) sites incorporated into the PCR primers, generating a construct pLOR67 containing the CDS for the fusion protein GAL4BD-N1 fagment 1762-1829. Diagnostic digests were constructed and for those constructs that appeared to contain the correct PCR fragment the insert cDNA and sub-cloning junctions were sequenced using primers PH41 and PH42 as follows.

| | |
|---|---|
| Forward (PH41-GAL4BDF) | |
| TCA TCG GAA GAG AGT AG | (SEQ ID NO: 37) |
| Reverse (PH42-GAL4BDR) | |
| CGT TTT AAA ACC TAA GAG TCA C | (SEQ ID NO: 38) |

The N1 fragment was sub-cloned into multi-cloning site 1 of pLOR32 (PBRIDGE vector from Clontech—address at 1 Becton Drive, Franklin Lakes, N.J. USA 07417—with ampicillin resistance substituted with kanamycin resistance), generating a construct pLOR67 containing the CDS for the fusion protein GAL4BD-N1 fragment 1762-1829 (FIG. 1B; SEQ ID NO: 3).

### Example 2

### Specificity Checks—Bait Construct

The ability of GAL4AD and various irrelevant GAL4AD-fusion proteins to activate reporter genes of AH109pLOR67 yeast strain was assessed by transforming the yeast with expression constructs for these proteins.

AH109 (Clontech) yeast strain was transformed with pLOR67 as follows:

10 ml YAPD (for AH 109) or 10 ml—W selection media (for yeast bait strains) were inoculated with the yeast strain to be transformed and grown at 30° C., shaking at 200 rpm overnight. Fresh media was then inoculated with the appropriate volume of the overnight culture to give an OD₆₀₀ reading of 0.2-0.3. This culture was allowed to grown until the yeast cells were in mid log phase growth and the OD₆₀₀ reading was 0.8-0.9. The yeast was pelleted by centrifugation, 4000 rpm for 5 minutes, washed once in sterile MQ-H₂O, twice in LiAc/TE and resuspended in 1 ml LiAc/TE per 100 ml original culture volume. 50 μl yeast suspension was added to 10 μl of DNA mix [0.2 μg plasmid DNA and 50 μg carrier DNA] and mixed well before 300 μl LiAc/TE/PEG solution was added. The transformation mix was gently mixed before being incubated at 30° C. for 30 minutes. The sample was then mixed again and incubated at 42° C. for 20 minutes. Transformed yeast was then washed in sterile MQ-H₂O, resuspended in 150 ml MQ-H₂O. The yeast was plated out on appropriate selection plates and these plates were incubated at 30° C. for 2-5 days until colonies were approximately 2-3 mm in diameter.

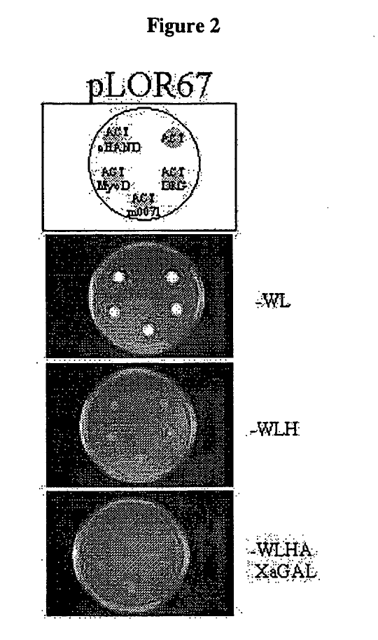

The transformants were plated on —WL (for verification of transformation) and —WLH, —WLHAXαGAL (for assessment of the expression of HIS3, ADE2 and LacZ reporter genes). FIG. 2 shows that the fusion protein GAL4BDN1(₁₇₆₂₋₁₈₂₉) does not autoactivate HIS3, ADE2 and LacZ reporter genes in the presence of the GAL4AD protein or irrelevant GAL4AD fusion proteins. Therefore the pLOR67 bait construct can be used to screen a GAL4AD cDNA library for proteins capable of interacting with the GAL4BD-N1 fragment (1762-1829) fusion protein.

### Example 3

### Yeast-Two-Hybrid Screen

A spleen cDNA library was selected as a means of investigating novel downstream components of the Notch1 receptor signalling pathways in immune reactions. AH109pLOR67 was transformed with human spleen cDNA library (Clontech).

AH109pLOR67 from Example 2 was transformed with a human spleen cDNA library (Clontech cat no. HL4054A1H, lot.8030674) as described below.

10 large—WLHA plates were prepared 2 days in advance of the transformation. 60 ml —W selection media was inoculated with the AH109pLOR67 yeast strain and grown at 30° C., shaking at 200 rpm over night. 500 ml fresh media was then inoculated with the appropriate volume (˜50 ml) of the overnight culture to give an OD₆₀₀ reading of 0.2-0.3. This culture was allowed to grow for approximately 3 hours until the yeast cells reached an OD₆₀₀ reading of 0.8-0.9. The yeast was pelleted by centrifugation, 4000 rpm for 5 minutes, washed once in sterile MQ-H₂O, and twice in LiAc/TE solution. The yeast pellet was then resuspended in 4 ml LiAc/TE solution. The DNA mix [100 μg library DNA in total volume of 200 μl, 400 μl of 50 μg/μl carrier DNA (Clontech), 75 μl 10×TE and 75 μl 1M LiAc] was mixed with the yeast suspension. 24 ml LiAc/TE/PEG solution was added to the yeast/DNA suspension, mixed by inversion, and incubated at 30° C. for 30 minutes. The transformation mix was then incubated at 42° C. for 20 minutes, the yeast was then pelleted by centrifugation, washed in MQ-H₂O and resuspended in 10 ml MQ-H₂O. 1 μl and 5 μl aliquots of the transformed yeast suspension were plated on —WL plates in order to determine the transformation efficiency of the yeast-two-hybrid screen. The remaining yeast suspension was divided equally between the 10 large—WLHA screen plates. All the plates were place in a 30° C. incubator and growth was assessed after 5 days. The number of colonies on each of the—WL plates was counted, and from this the total number of clones within the library screened in the yeast-two-hybrid screen was then determined. Yeast colonies capable of growing on —WLHA plates were transferred—WLHAXaGAL plates and their growth and expression of LacZ (blue colouration) were assessed over the following 48 hours.

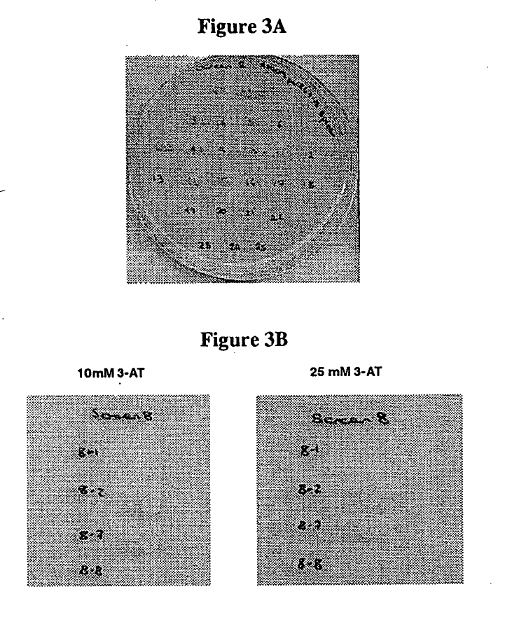

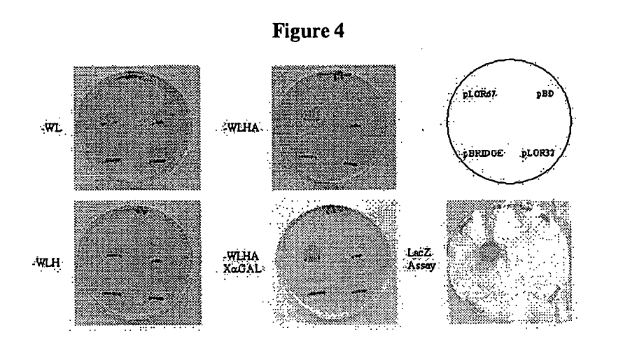

The yeast-two-hybrid screen had a transformation efficiency of 0.227 million and as the spleen library had a primary complexity of 3.5 million, this equates to 6.5% coverage of library complexity. Two colonies 8-2 and 8-7, selected on the—WLHA plates expressed lacZ (see FIG. 3A), and were capable of growing well under 3-amino-1, 2,4, -triazole selection, indicating strong protein interactions occurring in these yeast cells (see FIG. 3B). Initial identification of the GAL4AD fusion protein capable of interacting with the bait protein was undertaken. This was achieved by sequencing PCR products generated using the yeast cells as template and PCR primers PH27 and PH28 which amplify cDNA inserts in pACT library vector as follows:

Templates were prepared by inoculating a freshly grown yeast colony into 100 μl template solution and incubating at 37° C. for 4 hours, resuspending the yeast hourly.

Primers:
Forward (PH27)

(SEQ ID NO: 39)

CTA TTC GAT GAT GAA GAT ACC CCA CCA AAC CC
Reverse (PH28)

(SEQ ID NO: 40)

AAG TGA ACT TGC GGG GTT TTT CAG TAT CTA CG

| | |
|---|---|
| PCR reaction: | |
| Yeast template | 1 μl |
| 10× Buffer | 5 μl |
| PH27 | 1.5 μl |
| PH28 | 1.5 μl |
| dNTP's | 2 μl |
| MgCl₂ | 2 μl |
| Taq Hot Star | 0.5 μl |
| H₂O | 36.5 μl |
| PCR cycle: | |
| 95° C. 15 minutes | |
| 95° C. 30 seconds | |
| 50° C. 30 seconds | |
| 72° C. 3 minutes | |
| 72° C. 10 minutes | |
| 30 cycles | |

The PCR products were then sequenced by first cleaning up the reaction using Exo-SAP reaction, then sequencing the PCR product using ALA2 sequencing primer.

ExoI-SAP clean up:

| | |
|---|---|
| Yeast PCR | 30 μl |
| SAP | 1 μl |
| ExoI | 1 μl |
| SAP buffer | 4 μl |
| H₂O | 4 μl |

Incubated at 37° C. for 30 minutes and the enzymes denatured at 80° C. for 20 minutes.

| | | |
|---|---|---|
| Primer: | | |
| ALA2 | TTA CCC ATA CGA TGT TCC AG | (SEQ ID NO: 41) |

Sequence generated from these sequencing reactions was used to conduct a BLASTN search of the Genbank non-redundant database and the following were identified:

| Colony | Match | Accession Number |
|---|---|---|
| 8-2 | 0.0 | emb\|L07872\|HOMO sapiens |
| | | recombination signal binding |
| | | protein (CBF1/RBPJk) |
| 8-7 | 0.0 | emb\|AK000377\|HOMO sapiens |
| | | cDNA FLJ20370 fis, clone |
| | | HEP19443 |
| | | also known as C2PA |

The fact that a previously identified protein which is known to interact with the Notch 1 RAM domain (CBF1/RBPJk) has been isolated is consistent with the RAM domain of the Notch1 fragment folding correctly in the yeast.

### Example 4

### Specificity Checks—GAL4 Activation Domain (AD) Fusion Proteins

AH109pLOR67 and AH109 transformed with GAL4BD expression constructs pBD-GAL4cam (Stratagene), pBRIDGE (Clontech) and pLOR32 were used to assess whether the GAL4AD fusion proteins encoded by plasmids 8-2 and 8-7 interacted in a repeatable and specific manner with the Notch1(₁₇₆₂₋₁₈₂₉) domain of the GAL4BDN1(₁₇₆₂₋₁₈₂₉) fusion protein.

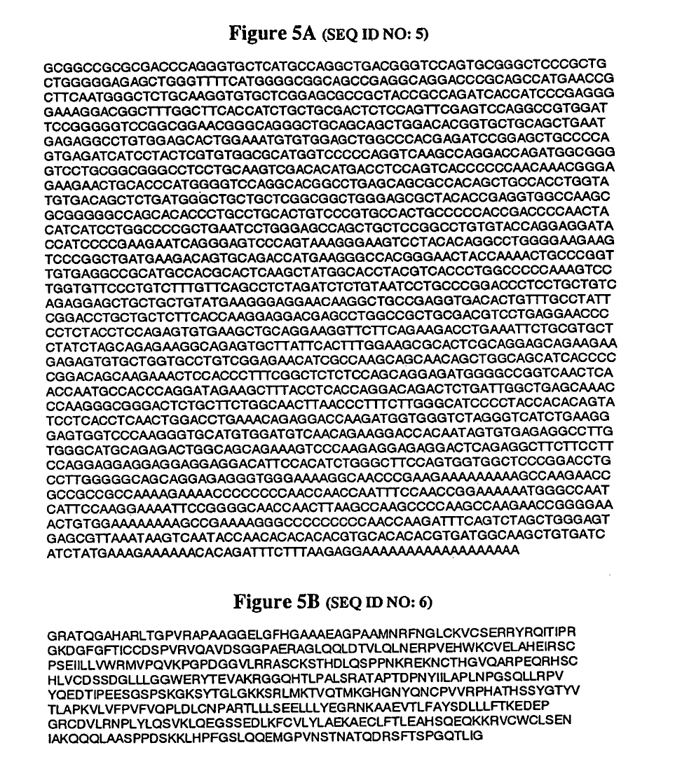

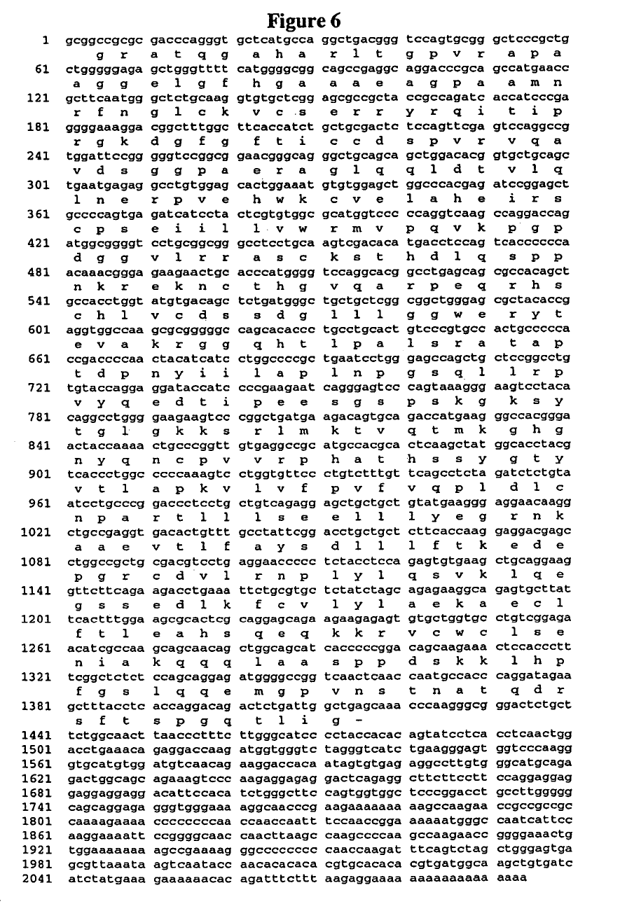

Yeast strain AH 109 was transformed (as described above) with GAL4BD expression constructs pBD-GAL4cam (Stratagene), pBRIDGE (Clontech) and pLOR32. These yeast stains along with AH109pLOR67 were then transformed with either 8-2 (CBF 1 fragment) or 8-7 and plated on —WL, —WLH, —WLHA, —WLHAXαGAL plates. An alternative to XaGAL was also employed to assess LacZ expression. Yeast colonies grown on —WLHA plates, were transferred to filter paper by placing the appropriately sized piece of 3 mm Whatman paper over the screen plate and pressing down firmly over the whole surface of the plate. The orientation of the paper was then marked and the paper lifted with a jerking action, lifting the colonies from the agar. The yeast colonies were permeabilised by placing the filter papers in liquid nitrogen, and then allowing them to thaw. This cycle was repeated three times in total. 1.8 ml Z buffer containing X-gal was added to each filter paper, the papers were then monitored for the next 6 hours for the production of blue colour. Results are shown in FIG. 5.

It can be seen from these results that interaction between the GAL4BD alone and GAL4AD fusion proteins did not occur, whereas activation of the HIS3, ADE2 and LacZ reporter genes due to protein-protein interactions repeatedly occurred in AH109pLOR67 when transformed with plasmids 8-2 & 8-7 (—WLH, —WLHA, —WLHAXαGAL and LacZ assay).

### Example 5

### Plasmid 8-7 Appears to Encode a Previously Unidentified Splice Variant of the C2PA Protein

The cDNA insert of plasmid 8-7 was fully sequenced as follows:

5 ml —WL selection media was inoculated with yeast colony and incubated at 30° C. in a shaking incubator for 2-3 days until the culture media was saturated. The yeast was pelleted by centrifugation, 2000 rpm for 5 minutes and resuspended in 300 μl Lyticase miniprep solution and incubated at 37° C. for 4 hours. The yeast cells were then lysed by the addition of 300 μl P2 solution (Qiagen), followed by incubation at 65° C. for 5 minutes. 300 μl P3 solution (Qiagen) was then added and the cellular debris was pelleted by centrifugation, 14,000 rpm for 20 minutes. The supernatant was transferred to a fresh eppendorf, 5 μl RNase A solution (10 mg/ml) was added and the solution incubated at room temperature for 5 minutes. Contaminating proteins, linear DNA and RNA was removed from the sample by adding 15 μl Strataclean resin (Stratagene), mixing the sample and allowing it to stand for at least 5 minutes. The resin and contaminants were removed by centrifugation, 14,000 rpm for 1 minute. The 800 μl supernatant was transferred to a fresh eppendorf and DNA was precipitated by the addition of 600 μl Propan-2-ol, and centrifugation 14,000 rpm for 30 minutes. The DNA pellet was washed in 70% ethanol and plasmid DNA resuspended in 40 μl MQ-H₂O. 1 μl plasmid DNA solution was used to transform by electroporation DH5α competent cells.

Transformed bacteria were plated out on LB-ampicillin plates and incubated overnight at 37° C. Plasmid DNA was extracted from bacterial colonies using Qiagen spin prep kits as protocol. Plasmid DNA was sequenced using vector specific primers PH35 and PH36 and insert specific primers PH122 to PH133 as follows:

| | |
|---|---|
| PH35-GAL4ADf: | |
| GTG CGA CAT CAT CAT CGG AAG | (SEQ ID NO: 42) |
| PH36-GAL4ADr: | |
| CCT AAG AGT CAC TTT AAA ATT | (SEQ ID NO: 43) |
| PH122-C2PA1f: | |
| GCC CCA GTG AGA TCA TCC TA | (SEQ ID NO: 44) |
| PH123-C2PA2f: | |
| CTG CAA GTC GAC ACA TGA CC | (SEQ ID NO: 45) |
| PH124-C2PA3f: | |
| GGA TAC CAT CCC CGA AGA AT | (SEQ ID NO: 46) |
| PH125-C2PA4f: | |
| TAT TCG GAC CTG CTG CTC TT | (SEQ ID NO: 47) |
| PH132-C2PA5f: | |
| GAG GAG GAG GAG GAC ATT CC | (SEQ ID NO: 48) |
| PH133-C2PA6f: | |
| CAG CAG AAA GTC CCA AGA GG | (SEQ ID NO: 49) |

A consensus sequence for the insert cDNA of plasmid 8-7 was generated by assembling the individual sequencing reactions in GAP4.

The resulting consensus sequence of 2094 bp which encodes in-frame with the upstream GAL4AD a 470 amino acid peptide. FIG. 5A (SEQ ID NO: 5) shows the nucleotide sequence of the fragment and FIG. 5B (SEQ ID NO: 6) shows the corresponding amino acid sequence.

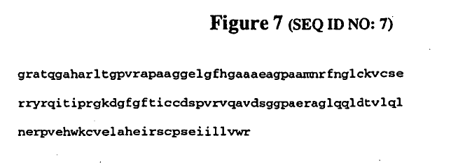

The identity of the insert cDNA was established using a BLASTN search of the GenBank non-redundant database. A large proportion of the insert cDNA is identical to the human homologue of mouse C2PA (GenBank Accession Numbers NM_{—}017790 and AK000377;), but BLAST searches of the human non-redundant and EST databases failed to find matches for the first 166 bp. BLASTN searches of the GENBANK high throughput human genomic database identified a clone (AL359455) that contained fragments of sequence that matched the human homologue of the C2PA gene and all of the first 166 bp cDNA insert encoded in plasmid 8-7. This suggests that the cDNA encoded by plasmid 8-7 may be a splice variant of the previously identified human homologue of mouse C2PA containing novel coding sequence at the amino-terminal region of the molecule (see FIG. 7; SEQ ID NO: 7).

### Example 6

### Identification of Possible Splicing Variants of the C2PA Gene

The sequence of the human C2PA gene was used as a BLAST probe on the GenBank database. This search identified the expected C2PA sequences deposited in the human division of the database and three highly homologous sequences in the mouse division of GenBank (see Figures).

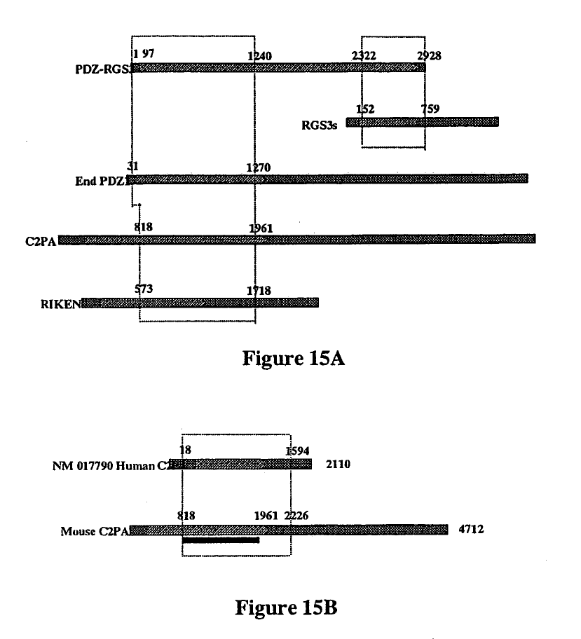

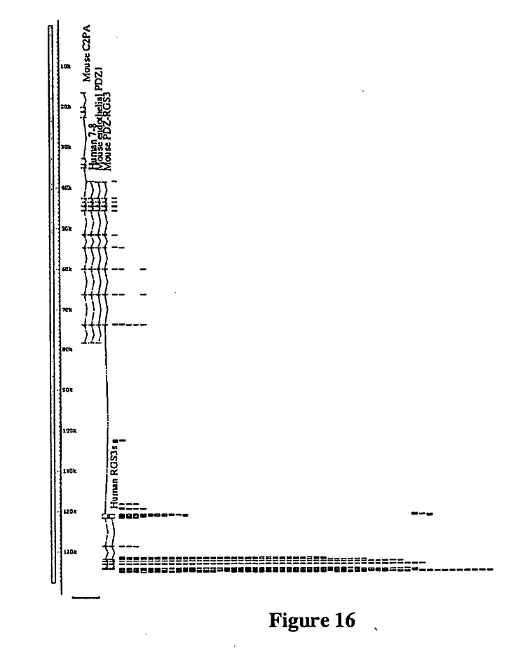

Without wishing to be bound by any theory, it is hypothesised that C2PA, the Riken cDNA, the endothelial PDZ protein and the PDZ-RGS3 genes are all splicing variants of the same transcriptional unit. FIG. 15A shows the result of the alignment between these genes and indicates that this hypothesis is correct, as they share a large portion of nucleotide identity in the upstream region containing the PDZ domain.

Further splicing variants outside the region of identity with C2PA were sought using the sequence of mouse PDZ-RGS3 as a BLAST probe on the GenBank database. This search identified the human RGS3 gene (AF215670, AF215669) as a splicing variant containing the RGS domain. Importantly the human C2PA gene ORF is conserved in the variants containing the PDZ domain which is consistent with interaction with the intracellular portion of the Notch receptor.

### Example 7

### Identification of Possible Human Splicing Variants of the C2PA Gene

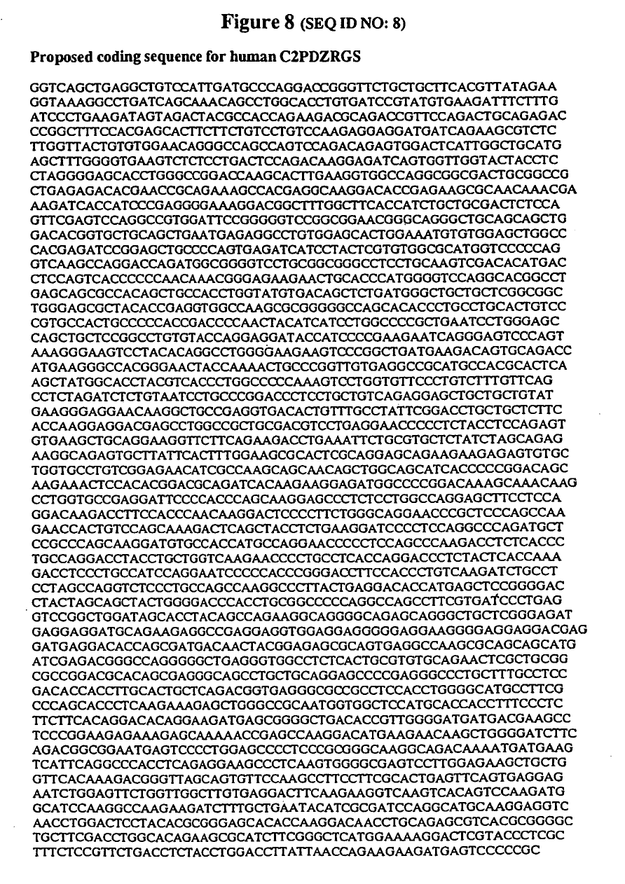

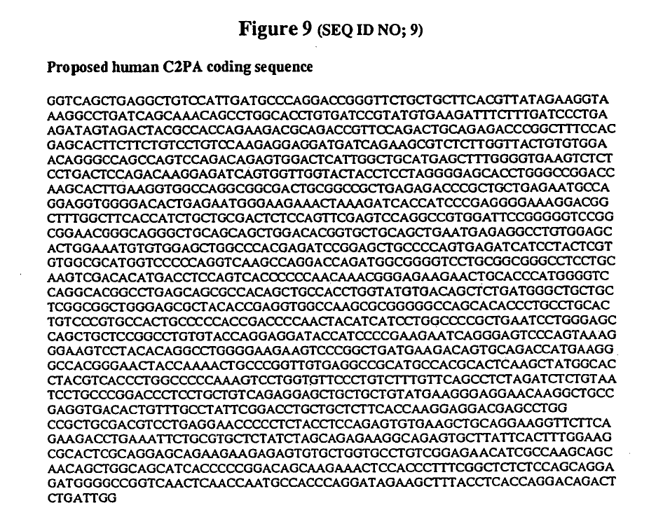

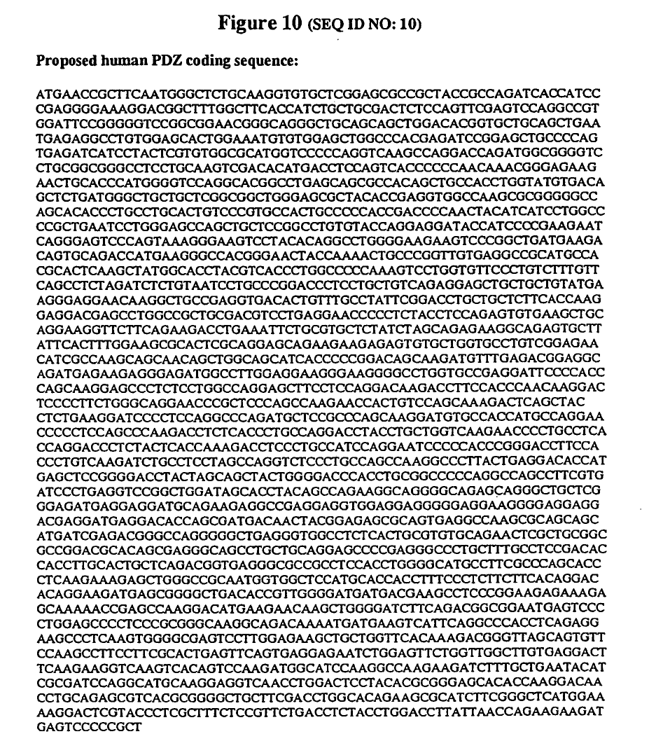

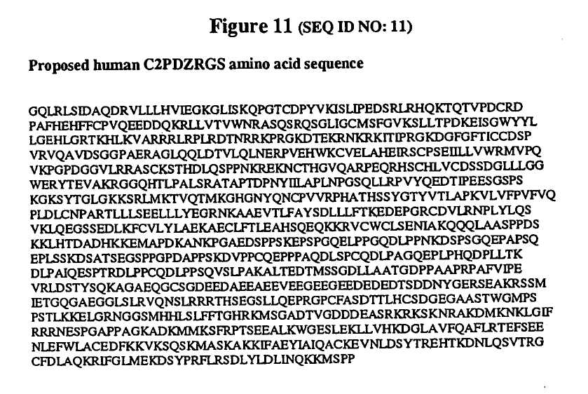

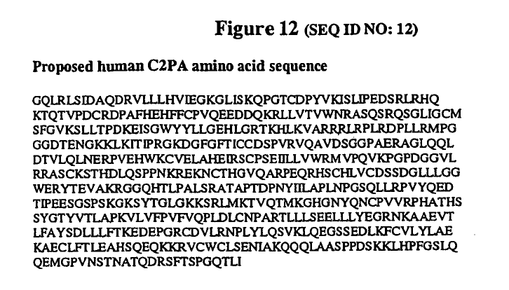

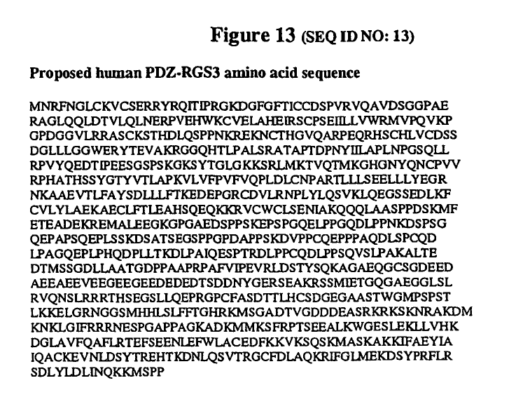

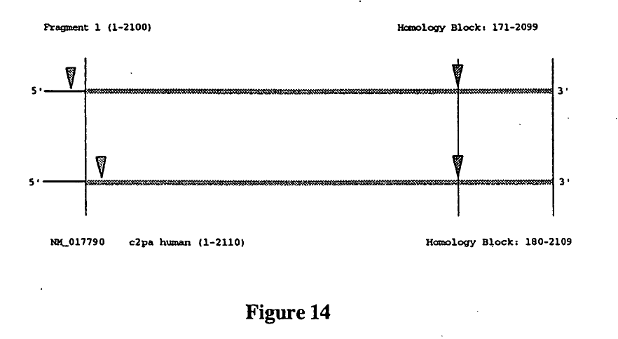

An attempt was made to map the mouse sequence on human genomic sequence data and derive the ORF. Searching for PACS revealed two contigs matching the query sequences and further analysis resulted in sequences for a proposed human gene C2PDZRGS and for proposed human homologues of the mouse genes C2PA and PDZ-RGS3 (see FIGS. 8 to 13; SEQ ID NOS: 8 to 13 respectively).

### Example 8

### Gene Expression Profiling

(i) hDelta1-IgG4Fc Fusion Protein

A fusion protein comprising the extracellular domain of human Delta1 fused to the Fc domain of human IgG4 (“hDelta1-IgG4Fc”) was prepared by inserting a nucleotide sequence coding for the extracellular domain of human Deltal (see, e.g. Genbank Accession No AF003522) into the expression vector pCONγ (Lonza Biologics, Slough, UK) and expressing the resulting construct in CHO cells. The amino acid sequence of the resulting expressed fusion protein was as follows (SEQ ID NO: 50):

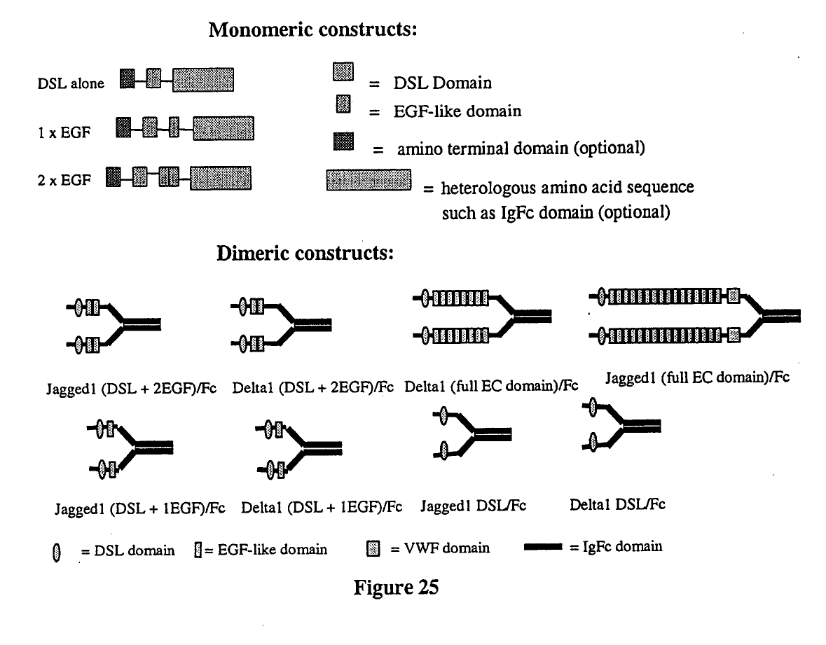

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERG
HGYVCECARGYGGPNCQFLLPELPPGPAVVDLTEKLEASTKGPSVFPLAP
CSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPE
FLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALH
NHYTQKSLSLSLGK

Wherein the first underlined sequence is the signal peptide (cleaved from the mature protein) and the second underlined sequence is the IgG4 Fc sequence. The protein normally exists as a dimer linked by disulphide bonds (see eg schematic representation in FIG. 25).

(ii) Cell Culture, Treatments and RNA Extraction

Freshly-isolated murine CD4⁺ T-cells were cultured in RPMI 1640 (GibcoBRL) supplemented with 2 mM Glutamine (GibcoBRL), Penicillin-Streptomycin 50 units/ml (GibcoBRL), 50 μM 2-mercaptoethanol and with 10% Fetal Bovine Serum (FBS) (Biochrom KG).

Anti-human IGg4 anti-CD3 (human), anti-CD28 (human) antibodies (PharMingen) were plated at 5 μg/ml in phosphate buffer saline (Gibco BRL) in 6 well tissue culture dishes (1 ml PBS/well) overnight. Anti-IgG4 antibody was applied to every well, while mouse IgG₁ κ isotype control at 10 μg/ml was applied in wells that no anti-CD3 or anti-CD28 was used. The next day the wells were washed 3 times with PBS, and human Delta1-IgG4Fc fusion protein (hDelta1-IgG4Fc; see above) was plated at 10 μg/ml in PBS (1 ml/well) upon ligand-treated wells: control wells were coated with PBS only. The plates were then incubated at 37° C. for 2 hours and then washed with PBS three times. CD4+ T-cells cells were then plated out at a concentration of 1×10⁶ cells/ml and incubated at 37° C. Cells were taken out after 4 hours and 16 hours (overnight), span down and lysed in 300-600111 RLT lysis solution (Qiagen). In order to ensure the efficacy of the Notch ligand stimulation, parallel cultures of cells were tested for expression of cytokines by standard ELISA techniques after 3 days in culture.

RNA was extracted using an RNA Easy miniprep kit (Qiagen) according to the manufacturer's instructions. The optional DNase step recommended was also performed. A phenol extraction step was performed to ensure the removal of proteins in the RNA. RNA was then amplified using the MessageAmp aRNA Kit (Ambion) following the manufacturer's recommendations. Briefly, the procedure consists of reverse transcription with an oligo(dT) primer bearing a T7 promoter and in vitro transcription of the resulting DNA with T7 RNA polymerase to generate hundreds of thousands of antisense RNA (αRNA) copies of each mRNA in the sample.

(iii) Gene Expression Profiling

Microarrays were manufactured by spotting purified PCR products onto glass slides. Microarray probes were prepared by labelling 2 μg of αRNA by a reverse transcriptase reaction incorporating dCTP-Cy3 or dCTP-Cy5 labelled nucleotide. Probe labelling and purification were then performed generally as described in Hegde P, Qi R, Abernathy K, Gay C, Dharap S, Gaspard R, Hughes J E, Snesrud E, Lee N, Quackenbush J: A concise guide to cDNA microarray analysis (2000). Biotechniques 29:548-50, 552-4, 556 passim.

Purified probes were then hybridized on the arrays overnight at 42° C. in 10×SSC, 50% formamide, 0.2% SDS solution. Slides were then washed twice in 2×SSC, 0.2% SDS for 7 min at 42° C., twice in 0.1 SSC/0.2% SDS for 5 minutes at room temperature, and finally once in 0.2% SSC at room temperature. For each time point the sample treated in the absence of Notch ligand (‘CD3/CD28 only’) was labelled with dCTP-Cy3 and hybridized on the same slide as the sample named ‘CD3/CD28 plus Delta’ that was labelled with dCTP-Cy5. Once dried the slides were scanned on a GSI Lumonics confocal scanner at 100% laser power and 65-75% photo-multiplier tube efficiency (depending on background). Slide images were processed as follows: Array spots representing the signal associated with individual spotted clones were identified and quantified using the Quantarray application (GSI Lumonics). Numeric values for the gene expression intensities were calculated using the histogram method implemented in the same application. Values were calculated as integrals of the pixel signal distribution associated to each spot and local background values subtracted (raw data).

(iv) Data Processing

For all data analyses the GeneSpring package (Silicon Genetics) was used. Raw data from Quantarray was introduced in GeneSpring, and the ratio between the signal and control intensities was calculated for each gene at each time point. Intensities for genes from samples labelled ‘CD3/CD28 plus Delta’ were regarded as ‘signals’ while the intensities from genes from samples labelled ‘CD3/CD28 only’ were regarded as ‘controls’.

Ratio=signal strength of gene in ‘CD3/CD28 plus Delta’/‘CD3/CD28 only’

When this ratio was >2 the gene was considered to be upregulated, while when the ratio was <0.5 the ratio the gene was considered to be down-regulated.

After 4 hours, SDF1 expression was found to be increased approximately 3-fold with CD3/CD28 plus Delta compared to CD3/CD28 only.

### Example 9

### ELISA Assay Method For Detecting Notch Signalling Activity

(i) CD4+ Cell Purification

Spleens were removed from mice (variously Balb/c females, 8-10 weeks, C57B/6 females, 8-10 weeks, D011.10 transgenic females, 8-10 weeks) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with DPBS plus 1 μg/ml anti-hamster IgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus hDelta1-IgG4Fc fusion protein (10 μg/ml; as described above) as modulator of Notch signalling. The plates were incubated for 2-3 hours at 37° C. then washed again with DPBS before cells (prepared as described above) were added.

(iii) Primary Polyclonal Stimulation and ELISA

CD4+ cells were cultured in 96 well, flat-bottomed plates pre-coated according to (ii) above. Cells were re-suspended, following counting, at 2×10⁶/ml in RIOF medium plus 4 μg/ml anti-CD28 antibody (Pharmingen, Cat No 553294, Clone No 37.51). 100 μl cell suspension was added per well. 100 μl of R1° F. medium was then added to each well to give a final volume of 200 μl (2×10⁵ cells/well, anti-CD28 final concentration 2 μg/ml). The plates were then incubated at 37° C. for 72 hours.

125 μl supernatant was then removed from each well and stored at −20° C. until tested by ELISA for IL-2, IL-10, IFN-γ and IL-13 using antibody pairs from R & D Systems (Abingdon, UK).

An increase in IL-10 expression and/or decrease in IFN-γ activity is indicative of Notch signalling activity.

Potential inhibitors may be added in the presence of, for example, hDelta1-IgG4Fc, to measure any inhibitory effects on Notch signalling.

The invention is further described by the following numbered paragraphs:
1. A method for modifying chemokine signalling by administering an effective amount of a modulator of the Notch signalling pathway.
2. A method for modifying chemokine signal transduction by administering an effective amount of a modulator of the Notch signalling pathway.
3. A method as described in paragraph 2 wherein the signal transduction is G-protein mediated.
4. A method as described any one of paragraphs 1-3 wherein the chemokine is SDF1
5. A method as described in any one of paragraphs 1 to 4 wherein chemokine signalling is inhibited.
6. A method as described in paragraph 5 wherein chemokine signalling is inhibited in immune cells.
7. A method as described in paragraph 6 for treating inflammation.
8. A method as described in any one of the preceding paragraphs wherein the modulator of the Notch signalling pathway comprises Delta or Jagged or a fragment, derivative, homologue, analogue or allelic variant thereof or a polynucleotide coding for Delta, Jagged or a fragment, derivative, homologue, analogue or allelic variant thereof.
9. A method as described in any one of paragraphs 1 to 7 wherein the modulator of the Notch signalling pathway comprises a fusion protein comprising a segment of a Notch ligand extracellular domain and an immunoglobulin Fc segment or a polynucleotide coding for such a fusion protein.
10. A method as described in any one of paragraphs 1 to 7 wherein the modulator of the Notch signalling pathway comprises a protein or polypeptide comprising a DSL or EGF-like domain or a fragment, derivative, homologue, analogue or allelic variant thereof or a polynucleotide sequence coding for such a protein, polypeptide, fragment, derivative, homologue, analogue or allelic variant.
11. A method as described in any one of paragraphs 1 to 7 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.
12. A method as described in any one of paragraphs 1 to 7 wherein the modulator of the Notch signalling pathway comprises a dominant negative version of a Notch signalling repressor, or a polynucleotide which codes for a dominant negative version of a Notch signalling repressor.
13. A method as described in any one of paragraphs 1 to 7 wherein the modulator of the Notch signalling pathway comprises a polypeptide capable of upregulating the expression or activity of a Notch ligand or a downstream component of the Notch signalling pathway, or a polynucleotide which codes for such a polypeptide.
14. A method as described in any one of paragraphs 1 to 7 wherein the modulator of the Notch signalling pathway comprises a polypeptide selected from Noggin, Chordin, Follistatin, Xnr3 and FGF or a fragment, derivative, homologue, analogue or allelic variant thereof, or a polynucleotide which codes for such a polypeptide, fragment, derivative, homologue, analogue or allelic variant.
15. A method as described in any one of paragraphs 1 to 7 wherein the modulator of the Notch signalling pathway comprises an immunosuppressive cytokine selected from IL-10, IL-13, TGF-β and FLT3 ligand or a fragment, derivative, homologue, analogue or allelic variant thereof, or a polynucleotide which codes for such an immunosuppressive cytokine, fragment, derivative, homologue, analogue or allelic variant.
16. The use of a modulator of the Notch signalling pathway in the manufacture of a medicament for modifying chemokine signalling.
17. The use of a modulator of the Notch signalling pathway in the manufacture of a medicament for modifying chemokine signal transduction.
18. The use of a modulator of the Notch signalling pathway in the manufacture of a medicament for the treatment of inflammation.
19. A use as described in any one of paragraphs 16 to 18 wherein the modulator of the Notch signalling pathway comprises Delta or Jagged or a fragment, derivative, homologue, analogue or allelic variant thereof or a polynucleotide coding for Delta, Jagged or a fragment, derivative, homologue, analogue or allelic variant thereof.
20. A use as described in any one of paragraphs 16 to 18 wherein the modulator of the Notch signalling pathway comprises a fusion protein comprising a segment of a Notch ligand extracellular domain and an immunoglobulin F_{c} segment or a polynucleotide coding for such a fusion protein.
21. A use as described in any one of paragraphs 16 to 18 wherein the modulator of the Notch signalling pathway comprises a protein or polypeptide comprising a DSL or EGF-like domain or a fragment, derivative, homologue, analogue or allelic variant thereof or a polynucleotide sequence coding for such a protein, polypeptide, fragment, derivative, homologue, analogue or allelic variant.
22. A use as described in any one of paragraphs 16 to 18 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.
23. A use as described in any one of paragraphs 16 to 18 wherein the modulator of the Notch signalling pathway comprises a dominant negative version of a Notch signalling repressor, or a polynucleotide which codes for a dominant negative version of a Notch signalling repressor.
24. A use as described in any one of paragraphs 16 to 18 wherein the modulator of the Notch signalling pathway comprises a polypeptide capable of upregulating the expression or activity of a Notch ligand or a downstream component of the Notch signalling pathway, or a polynucleotide which codes for such a polypeptide.
25. A use as described in any one of paragraphs 16 to 18 wherein the modulator of the Notch signalling pathway comprises a polypeptide selected from Noggin, Chordin, Follistatin, Xnr3 and FGF or a fragment, derivative, homologue, analogue or allelic variant thereof, or a polynucleotide which codes for such a polypeptide, fragment, derivative, homologue, analogue or allelic variant.
26. A use as described in any one of paragraphs 16 to 18 wherein the modulator of the Notch signalling pathway comprises an immunosuppressive cytokine selected from IL-10, IL-13, TGF-β and FLT3 ligand or a fragment, derivative, homologue, analogue or allelic variant thereof, or a polynucleotide which codes for such an immunosuppressive cytokine, fragment, derivative, homologue, analogue or allelic variant.
27. A method of modulating the trafficking of leukocytes, the method comprising: administering an effective amount of a modulator of Notch signalling, in a dose effective to modulate said trafficking of said leukocytes.
28. A method of enhancing migration of cells by administering a modulator of Notch signalling in an amount effective to enhance migration of cells.
29. A method of enhancing migration of cells to a site in a subject comprising locally administering to said site a modulator of Notch signalling in an amount effective to enhance migration of cells to the site in the subject.
30. A method of inhibiting migration of cells comprising administering a modulator of Notch signalling in an amount effective to inhibit migration of cells.
31. A method of inhibiting migration of cells to a site in a subject comprising locally administering to said site a modulator of Notch signalling in an amount effective to inhibit migration of cells to the site in the subject.
32. A method as described wherein the cells are immune cells, neural cells, epithelial cells, endothelial cells or mesenchymal cells.
33. A method of repelling cells from a material surface comprising coating a material surface with an amount of a modulator of Notch signalling effective to repel cells from the material surface.
34. A method of attracting cells to a material surface comprising coating a material surface with a modulator of Notch signalling in an effective amount to attract cells to the material surface.
35. A method of enhancing an immune response in a subject having a condition that involves defective chemokine signalling.
36. A method for inhibiting angiogenesis by administering a modulator of Notch signalling.
37. A method for increasing angiogenesis by administering a modulator of Notch signalling.
38. A method for modifying ephrin signalling by administering an effective amount of a modulator of the Notch signalling pathway.
39. A method of altering the sensitivity of a cell to a chemokine by administering a modulator of Notch signalling.
40. A method as described in paragraph 39 for modifying cell migration, blood vessel formation, or axon pathway selection.
41. A method for modifying cell migration by administering an effective amount of a modulator of the Notch signalling pathway.
42. A method for modifying cellular extravasion by administering an effective amount of a modulator of the Notch signalling pathway.
43. A polypeptide comprising an amino acid sequence which has at least 95% identity to the amino acid sequence of SEQ ID NO: 6, over its entire length.
44. A polypeptide according to paragraph 43 comprising the amino acid sequence of SEQ ID NO: 6.
45. A polynucleotide comprising a nucleotide sequence encoding a polypeptide which has at least 95% identity to the amino acid sequence of SEQ ID NO: 6, over the entire length of SEQ ID NO: 6, or a nucleotide sequence fully complementary to said polynucleotide.
46. A polynucleotide having the sequence of SEQ ID NO: 5.
47. A polypeptide comprising an amino acid sequence which has at least 80% identity to the amino acid sequence of SEQ ID NO: 7, over the entire length of SEQ ID NO: 7, or a fragment, variant or homolog thereof.
48. A polypeptide according to paragraph 47 comprising the amino acid sequence of SEQ ID NO: 7.
49. A polynucleotide comprising a nucleotide sequence encoding a polypeptide which has at least 80% identity to the amino acid sequence of SEQ ID NO: 7, over the entire length of SEQ ID NO: 7, or a nucleotide sequence fully complementary to said polynucleotide.
50. A polynucleotide which hybridises to a nucleotide sequence encoding a polypeptide which has at least 80% identity to the amino acid sequence of SEQ ID NO:7, over the entire length of SEQ ID NO: 7.
51. A polynucleotide comprising a nucleotide sequence encoding the polypeptide of SEQ ID NO: 7 or obtainable by screening an appropriate library under stringent hybridisation conditions with a probe.
52. A polynucleotide comprising a nucleotide sequence which has at least 80% identity to the sequence of SEQ ID NO: 8, over the entire length of SEQ ID NO: 8, or a nucleotide sequence fully complementary to said polynucleotide.
53. A polynucleotide comprising a nucleotide sequence which has at least 80% identity to the sequence of SEQ ID NO: 9, over the entire length of SEQ ID NO: 9, or a nucleotide sequence fully complementary to said polynucleotide.
54. A polynucleotide comprising a nucleotide sequence which has at least 80% identity to the sequence of SEQ ID NO: 10, over the entire length of SEQ ID NO: 10, or a nucleotide sequence fully complementary to said polynucleotide.
55. A polypeptide comprising an amino acid sequence which has at least 80% identity to the amino acid sequence of SEQ ID NO: 11, over the entire length of SEQ ID NO: 11, or a fragment, variant or homolog thereof.
56. A polypeptide according to paragraph 55 comprising the amino acid sequence of SEQ ID NO: 11.
57. A polynucleotide comprising a nucleotide sequence encoding a polypeptide which has at least 80% identity to the amino acid sequence of SEQ ID NO: 11, over the entire length of SEQ ID NO: 11, or a nucleotide sequence fully complementary to said polynucleotide.
58. A polynucleotide which hybridises to a nucleotide sequence encoding a polypeptide which has at least 80% identity to the amino acid sequence of SEQ ID NO: 11, over the entire length of SEQ ID NO: 11.
59. A polynucleotide comprising a nucleotide sequence encoding the polypeptide of SEQ ID NO: 11 or obtainable by screening an appropriate library under stringent hybridisation conditions with a probe.
60. A polypeptide comprising an amino acid sequence which has at least 80% identity to the amino acid sequence of SEQ ID NO: 12, over the entire length of SEQ ID NO: 12, or a fragment, variant or homolog thereof.
61. A polypeptide according to paragraph 60 comprising the amino acid sequence of SEQ ID NO: 12.
62. A polynucleotide comprising a nucleotide sequence encoding a polypeptide which has at least 80% identity to the amino acid sequence of SEQ ID NO: 12, over the entire length of SEQ ID NO: 12, or a nucleotide sequence fully complementary to said polynucleotide.
63. A polynucleotide which hybridises to a nucleotide sequence encoding a polypeptide which has at least 80% identity to the amino acid sequence of SEQ ID NO: 12, over the entire length of SEQ ID NO: 12.
64. A polynucleotide comprising a nucleotide sequence encoding the polypeptide of SEQ ID NO: 12 or obtainable by screening an appropriate library under stringent hybridisation conditions with a probe.
65. A polypeptide comprising an amino acid sequence which has at least 80% identity to the amino acid sequence of SEQ ID NO: 13, over the entire length of SEQ ID NO: 13, or a fragment, variant or homolog thereof.
66. A polypeptide according to paragraph 65 comprising the amino acid sequence of SEQ ID NO: 13.
67. A polynucleotide comprising a nucleotide sequence encoding a polypeptide which has at least 80% identity to the amino acid sequence of SEQ ID NO: 13, over the entire length of SEQ ID NO: 13, or a nucleotide sequence fully complementary to said polynucleotide.
68. A polynucleotide which hybridises to a nucleotide sequence encoding a polypeptide which has at least 80% identity to the amino acid sequence of SEQ ID NO: 13, over the entire length of SEQ ID NO: 13.
69. A polynucleotide comprising a nucleotide sequence encoding the polypeptide of SEQ ID NO: 13 or obtainable by screening an appropriate library under stringent hybridisation conditions with a probe.
70. A vector comprising a polynucleotide according to any one of paragraphs 45, 46, 49, 50, 51, 52, 53, 54, 57, 58, 59, 62, 63 or 64.
71. The vector of paragraph 70 wherein said polynucleotide is operatively linked to an expression control DNA sequence.
72. A host cell stably transformed or transfected with a polynucleotide according to any one of paragraphs 45, 46, 49, 50, 51, 52, 53, 54, 57, 58, 59, 62, 63 or 64 in a manner allowing the expression in said host cell of the polypeptide of paragraph 65.
73. A method for producing the polypeptide of any one of paragraphs 42, 44, 47, 48, 55, 56, 60, 61, 65, 66, 67, 68 or 69, said method comprising growing a host cell according to paragraph 72 in a suitable nutrient medium and isolating the polypeptide.
74. A method of identifying a compound that is a modulator of the polypeptide of any one of paragraphs 42, 44, 47, 48, 55, 56, 60, 61, 65, 66, 67, 68 or 69 or the polynucleotide of any one of paragraphs 45, 46, 49, 50, 51, 52, 53, 54, 57, 58, 59, 62, 63 or 64 comprising the steps of:
a) determining the activity of said polypeptide in the presence and absence of said compound;
b) comparing the activities observed in step (a); and
c) identifying said compound as a modulator by the observed differences in the activity of said polypeptide in the presence and absence of said compound.
75. A method according to paragraph 74 wherein the activity is observed via modulation of the Notch signalling pathway.
76. A modulator of the polypeptide of any one of paragraphs 42, 44, 47, 48, 55, 56, 60, 61, 65, 66, 67, 68 or 69 or the polynucleotide of any one of paragraphs 45, 46, 49, 50, 51, 52, 53, 54, 57, 58, 59, 62, 63 or 64 identifiable using the method of paragraph 74 or paragraph 75.
77. A polypeptide or peptide capable of specifically binding to the polypeptide of any one of paragraphs 42, 44, 47, 48, 55, 56, 60, 61, 65, 66, 67, 68 or 69.
78. A polypeptide or peptide as described in paragraph 77 which is an antibody.
79. An antibody as described in paragraph 78 which is a monoclonal antibody.
80. A hybridoma cell line producing a monoclonal antibody according to paragraph 79.
81. A pharmaceutical composition comprising the polypeptide of any one of paragraphs 42, 44, 47, 48, 55, 56, 60, 61, 65, 66, 67, 68 or 69, the polynucleotide of any one of paragraphs 45, 46, 49, 50, 51, 52, 53, 54, 57, 58, 59, 62, 63 or 64, or the modulator of paragraph 76 and a pharmaceutically acceptable diluent, carrier or excipient.

### REFERENCES

Altman J D et al Science 1996 274: 94-6.
Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Chee M. et al. (1996) Science 274:601-614.
Dkuz et al (1997) Cell 90: 271-280.
Hemmati-Brivanlou and Melton (1997) Cell 88:13-17.
Hicks C, et al. (2000) Nat. Cell. Biol. 2:515-520.
Iemura et al. (1998) PNAS 95:9337-9345.
Irvine K D (1999) Curr. Opin. Genet. Devel. 9:434-441.
Ju B J, et al. (2000) Nature 405:191-195.
Leimeister C. et al. (1999) Mech Dev 85 (1-2):173-7.
Li et al. (1998) Immunity 8 (1):43-55.
Lieber, T. et al. (1993) Genes Dev 7 (10):1949-65.
Lu, F. M. et al. (1996) Proc Natl Acad Sci 93 (11):5663-7.
Matsuno K, et al. (1998) Nat. Genet. 19:74-78.
Matsuno, K. et al. (1995) Development 121 (8):2633-44.
McGuinness T. Et al (1996) Genomics 35 (3):473-85.
Medhzhitov et al. (1997) Nature 388:394-397.
Meuer S. et al (2000) Rapid Cycle Real-time PCR, Springer-Verlag Berlin and Heidelberg GmbH & Co.
Moloney D J, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sasai et al. (1994) Cell 79:779-790.
Schroeter E H, et al. (1998) Nature 393:382-386.
Schroeter, E. H. et al. (1998) Nature 393 (6683):382-6.
Struhl G, Adachi A. (1998) Cell 93:649-660.
Struhl, G. et al. (1998) Cell 93 (4):649-60.
Takebayashi K. et al. (1994) J Biol Chem 269 (7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A method for modifying chemokine signalling in a target cell comprising administering an effective amount of a modulator of the Notch signalling pathway to the target cell.

2. The method as claimed in claim 1, wherein the signalling is G-protein mediated.

3. The method as claimed in claim 1, wherein the chemokine is stromal cell-derived factor (SDF1).

4. The method as claimed in claim 1, wherein chemokine signalling is inhibited.

5. The method as claimed in claim 4, wherein the target cell is an immune cell.

6. The method as claimed in claim 5, wherein the target cell is in a subject and wherein inflammation is treated in the subject.

7. The method as claimed in claim 1, wherein the modulator of the Notch signalling pathway is selected from the group consisting of: (i) Delta; (ii) Jagged; (iii) a fusion protein comprising a segment of a Notch ligand extracellular domain and an immunoglobulin F_{c} segment; (iv) a protein or polypeptide comprising a DSL domain; (v) a protein or polypeptide comprising an EGF-like domain; (vi) a protein or polypeptide comprising a Notch intracellular domain (Notch IC); (vii) a dominant negative version of a Notch signalling repressor; (viii) a polypeptide capable of upregulating the expression or activity of a Notch ligand or a downstream component of the Notch signalling pathway; (xi) Noggin; (x) Chordin; (xi) Follistatin; (xii) Xnr3; (xiii) FGF; (xiv) an immunosuppressive cytokine; (xv) a fragment, derivative, homologue, analogue or allelic variant thereof; and (xvi) a polynucleotide encoding therefor.

8. The method as claimed in 7, wherein the immunosuppressive cytokine is selected from the group consisting of IL-10, IL-13, TGF-β and FLT3 ligand or a fragment, derivative, homologue, analogue or allelic variant thereof, or a polynucleotide which codes for such an immunosuppressive cytokine, fragment, derivative, homologue, analogue or allelic variant.

9. A method of modulating migration of cells by administering a modulator of Notch signalling in an amount effective to modulate migration of cells.

10. The method as claimed in claim 9, wherein migration of cells is enhanced compared with untreated cells.

11. The method as claimed in claim 10, wherein migration of cells to a site in a subject is enhanced by locally administering the modulator of Notch signalling to the site in the subject.

12. The method as claimed in claim 9, wherein migration of cells is inhibited compared with untreated cells.

13. The method as claimed in claim 12, wherein migration of cells to a site in a subject is inhibited by locally administering the modulator of Notch signalling to the site in the subject.

14. The method as claimed in claim 9, wherein the cells are leukocytes and wherein trafficking of the leukocytes is modulated.

15. The method as claimed in claim 9, wherein the migration of cells is cellular extravasion.

16. The method as claimed in claim 9, wherein the cells are immune cells, neural cells, epithelial cells, endothelial cells or mesenchymal cells.

17. An isolated polynucleotide having the function of modifying chemoattraction, which polynucleotide:
a) has a nucleotide sequence of SEQ ID NO: 5;
b) comprises a nucleotide sequence encoding a polypeptide having at least about 95% identity to SEQ ID NO: 6, over the entire length of SEQ ID NO: 6;
c) hybridises under stringent conditions to the polynucleotide of a) or b); or
d) is fully complementary to the nucleotide sequence of a), b) or c).

18. An isolated polypeptide encoded by the polynucleotide as claimed in claim 17.

19. The polypeptide according to claim 18 having an amino acid sequence of SEQ ID NO: 6.

20. An isolated polynucleotide having the function of modifying chemoattraction, which polynucleotide:
a) encodes a polypeptide having at least about 80% identity to SEQ ID NO: 7, over the entire length of SEQ ID NO: 7,
b) hybridises under stringent conditions to the polynucleotide of a);
c) is fully complementary to the polynucleotide of a) or b).

21. An isolated polypeptide encoded by the polynucleotide as claimed in claim 20.

22. The polypeptide as claimed in claim 21 having an amino acid sequence of SEQ ID NO: 7.

23. An isolated polynucleotide having the function of modifying chemoattraction, which polynucleotide:
a) encodes a polypeptide having at least about 80% identity to SEQ ID NO: 8, over the entire length of SEQ ID NO: 8,
b) hybridises under stringent conditions to the polynucleotide of a);
c) is fully complementary to the polynucleotide of a) or b).

24. An isolated polynucleotide having the function of modifying chemoattraction, which polynucleotide:
a) encodes a polypeptide having at least about 80% identity to SEQ ID NO: 9, over the entire length of SEQ ID NO: 9,
b) hybridises under stringent conditions to the polynucleotide of a);
c) is fully complementary to the polynucleotide of a) or b).

25. An isolated polynucleotide having the function of modifying chemoattraction, which polynucleotide:
a) encodes a polypeptide having at least about 80% identity to SEQ ID NO: 10, over the entire length of SEQ ID NO: 10,
b) hybridises under stringent conditions to the polynucleotide of a);
c) is fully complementary to the polynucleotide of a) or b).

26. An isolated polynucleotide having the function of modifying chemoattraction, which polynucleotide:
a) encodes a polypeptide having at least about 80% identity to SEQ ID NO: 11, over the entire length of SEQ ID NO: 11,
b) hybridises under stringent conditions to the polynucleotide of a);
c) is fully complementary to the polynucleotide of a) or b).

27. An isolated polypeptide encoded by the polynucleotide as claimed in claim 26.

28. The polypeptide as claimed in claim 27 having an amino acid sequence of SEQ ID NO: 11.

29. An isolated polynucleotide having the function of modifying chemoattraction, which polynucleotide:
a) encodes a polypeptide having at least about 80% identity to SEQ ID NO: 12, over the entire length of SEQ ID NO: 12,
b) hybridises under stringent conditions to the polynucleotide of a);
c) is fully complementary to the polynucleotide of a) or b).

30. An isolated polypeptide encoded by the polynucleotide as claimed in claim 29.

31. The polypeptide as claimed in claim 30 having an amino acid sequence of SEQ ID NO:12.

32. An isolated polynucleotide having the function of modifying chemoattraction, which polynucleotide:
a) encodes a polypeptide having at least about 80% identity to SEQ ID NO: 13, over the entire length of SEQ ID NO: 13,
b) hybridises under stringent conditions to the polynucleotide of a);
c) is fully complementary to the polynucleotide of a) or b).

33. An isolated polypeptide encoded by the polynucleotide as claimed in claim 32.

34. The polypeptide as claimed in claim 33 having an amino acid sequence of SEQ ID NO:13.


SEQUENCE LISTING (54 sequences)

SEQ ID NO: 1 - protein, 101 aa, Artificial
  Notch1
FVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLD

SEQ ID NO: 2 - protein, 85 aa, Artificial
  protein aligned with Notch1 in fig2
EFRRQHGQLWFPEGFKVSEASKKKRREPLGEDSVGLKPLKNASDGALMDDNQNEWGDEDLETKKFRFEDPSTCSQANSGRISYDL

SEQ ID NO: 3 - DNA, 660 bp, Artificial
  Notch 1 fragment coding sequence
aagaattttcgttttaaaacctaagagtcactttaaaatttggattcacttattttttttataacttatttaataataaaaatcataaatcataagaaattcgcccggaattagcttggctgcaggtcgacggatcctcgaaccggaacttcttggtctccaggtcctcgtccccccactcattctggttgtcgtccatgagggcaccgtctgaagcgttcttcaggggcttgaggcccacggagtcctcgccgaggggctcccgccgcttcttcttgctggcctcagacactttgaagccctcagggaaccagagctggccatgctgccgccggaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgg

SEQ ID NO: 4 - protein, 233 aa, Artificial
  GAL4BD-N1
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFRRQHGQLWFPEGFKVSEASKKKRREPLGEDSVGLKPLKNASDGALMDDNQNEWGDEDLETKKFRFEDPSTCSQANSGRISYDL

SEQ ID NO: 5 - DNA, 2094 bp, Artificial
  cDNA insert
gcggccgcgcgacccagggtgctcatgccaggctgacgggtccagtgcgggctcccgctgctgggggagagctgggttttcatggggcggcagccgaggcaggacccgcagccatgaaccgcttcaatgggctctgcaaggtgtgctcggagcgccgctaccgccagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagaaactccaccctttcggctctctccagcaggagatggggccggtcaactcaaccaatgccacccaggatagaagctttacctcaccaggacagactctgattggctgagcaaacccaagggcgggactctgcttctggcaacttaaccctttcttgggcatcccctaccacacagtatcctcacctcaactggacctgaaacagaggaccaagatggtgggtctagggtcatctgaagggagtggtcccaagggtgcatgtggatgtcaacagaaggaccacaatagtgtgagaggccttgtgggcatgcagagactggcagcagaaagtcccaagaggagaggactcagaggcttcttccttccaggaggaggaggaggaggacattccacatctgggcttccagtggtggctcccggacctgccttgggggcagcaggagagggtgggaaaaggcaacccgaagaaaaaaaaagccaagaaccgccgccgccaaaagaaaaccccccccaaccaaccaatttccaaccggaaaaaatgggccaatcattccaaggaaaattccggggcaaccaacttaagccaagccccaagccaagaaccggggaaactgtggaaaaaaaagccgaaaagggcccccccccaaccaagatttcagtctagctgggagtgagcgttaaataagtcaataccaacacacacacgtgcacacacgtgatggcaagctgtgatcatctatgaaagaaaaaacacagatttctttaagaggaaaaaaaaaaaaaaaaaa

SEQ ID NO: 6 - protein, 470 aa, Artificial
  Peptide sequence coded by cDNA insert
GRATQGAHARLTGPVRAPAAGGELGFHGAAAEAGPAAMNRFNGLCKVCSERRYRQITIPRGKDGFGFTICCDSPVRVQAVDSGGPAERAGLQQLDTVLQLNERPVEHWKCVELAHEIRSCPSEIILLVWRMVPQVKPGPDGGVLRRASCKSTHDLQSPPNKREKNCTHGVQARPEQRHSCHLVCDSSDGLLLGGWERYTEVAKRGGQHTLPALSRATAPTDPNYIILAPLNPGSQLLRPVYQEDTIPEESGSPSKGKSYTGLGKKSRLMKTVQTMKGHGNYQNCPVVRPHATHSSYGTYVTLAPKVLVFPVFVQPLDLCNPARTLLLSEELLLYEGRNKAAEVTLFAYSDLLLFTKEDEPGRCDVLRNPLYLQSVKLQEGSSEDLKFCVLYLAEKAECLFTLEAHSQEQKKRVCWCLSENIAKQQQLAASPPDSKKLHPFGSLQQEMGPVNSTNATQDRSFTSPGQTLIG

SEQ ID NO: 7 - protein, 130 aa, Artificial
  Novel peptide coded by plasmid 8-7
GRATQGAHARLTGPVRAPAAGGELGFHGAAAEAGPAAMNRFNGLCKVCSERRYRQITIPRGKDGFGFTICCDSPVRVQAVDSGGPAERAGLQQLDTVLQLNERPVEHWKCVELAHEIRSCPSEIILLVWR

SEQ ID NO: 8 - DNA, 3178 bp, Homo sapiens
ggtcagctgaggctgtccattgatgcccaggaccgggttctgctgcttcacgttatagaaggtaaaggcctgatcagcaaacagcctggcacctgtgatccgtatgtgaagatttctttgatccctgaagatagtagactacgccaccagaagacgcagaccgttccagactgcagagacccggctttccacgagcacttcttctgtcctgtccaagaggaggatgatcagaagcgtctcttggttactgtgtggaacagggccagccagtccagacagagtggactcattggctgcatgagctttggggtgaagtctctcctgactccagacaaggagatcagtggttggtactacctcctaggggagcacctgggccggaccaagcacttgaaggtggccaggcggcgactgcggccgctgagagacacgaaccgcagaaagccacgaggcaaggacaccgagaagcgcaacaaacgaaagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagaaactccacacggacgcagatcacaagaaggagatggccccggacaaagcaaacaagcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaaccgagccaaggacatgaagaacaagctggggatcttcagacggcggaatgagtcccctggagcccctcccgcgggcaaggcagacaaaatgatgaagtcattcaggcccacctcagaggaagccctcaagtggggcgagtccttggagaagctgctggttcacaaagacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgc

SEQ ID NO: 9 - DNA, 1727 bp, Homo sapiens
ggtcagctgaggctgtccattgatgcccaggaccgggttctgctgcttcacgttatagaaggtaaaggcctgatcagcaaacagcctggcacctgtgatccgtatgtgaagatttctttgatccctgaagatagtagactacgccaccagaagacgcagaccgttccagactgcagagacccggctttccacgagcacttcttctgtcctgtccaagaggaggatgatcagaagcgtctcttggttactgtgtggaacagggccagccagtccagacagagtggactcattggctgcatgagctttggggtgaagtctctcctgactccagacaaggagatcagtggttggtactacctcctaggggagcacctgggccggaccaagcacttgaaggtggccaggcggcgactgcggccgctgagagacccgctgctgagaatgccaggaggtggggacactgagaatgggaagaaactaaagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagaaactccaccctttcggctctctccagcaggagatggggccggtcaactcaaccaatgccacccaggatagaagctttacctcaccaggacagactctgattgg

SEQ ID NO: 10 - DNA, 2750 bp, Homo sapiens
atgaaccgcttcaatgggctctgcaaggtgtgctcggagcgccgctaccgccagatcaccatcccgaggggaaaggacggctttggcttcaccatctgctgcgactctccagttcgagtccaggccgtggattccgggggtccggcggaacgggcagggctgcagcagctggacacggtgctgcagctgaatgagaggcctgtggagcactggaaatgtgtggagctggcccacgagatccggagctgccccagtgagatcatcctactcgtgtggcgcatggtcccccaggtcaagccaggaccagatggcggggtcctgcggcgggcctcctgcaagtcgacacatgacctccagtcaccccccaacaaacgggagaagaactgcacccatggggtccaggcacggcctgagcagcgccacagctgccacctggtatgtgacagctctgatgggctgctgctcggcggctgggagcgctacaccgaggtggccaagcgcgggggccagcacaccctgcctgcactgtcccgtgccactgcccccaccgaccccaactacatcatcctggccccgctgaatcctgggagccagctgctccggcctgtgtaccaggaggataccatccccgaagaatcagggagtcccagtaaagggaagtcctacacaggcctggggaagaagtcccggctgatgaagacagtgcagaccatgaagggccacgggaactaccaaaactgcccggttgtgaggccgcatgccacgcactcaagctatggcacctacgtcaccctggcccccaaagtcctggtgttccctgtctttgttcagcctctagatctctgtaatcctgcccggaccctcctgctgtcagaggagctgctgctgtatgaagggaggaacaaggctgccgaggtgacactgtttgcctattcggacctgctgctcttcaccaaggaggacgagcctggccgctgcgacgtcctgaggaaccccctctacctccagagtgtgaagctgcaggaaggttcttcagaagacctgaaattctgcgtgctctatctagcagagaaggcagagtgcttattcactttggaagcgcactcgcaggagcagaagaagagagtgtgctggtgcctgtcggagaacatcgccaagcagcaacagctggcagcatcacccccggacagcaagatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaaccgagccaaggacatgaagaacaagctggggatcttcagacggcggaatgagtcccctggagcccctcccgcgggcaaggcagacaaaatgatgaagtcattcaggcccacctcagaggaagccctcaagtggggcgagtccttggagaagctgctggttcacaaagacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgct

SEQ ID NO: 11 - protein, 1059 aa, Homo sapiens
GQLRLSIDAQDRVLLLHVIEGKGLISKQPGTCDPYVKISLIPEDSRLRHQKTQTVPDCRDPAFHEHFFCPVQEEDDQKRLLVTVWNRASQSRQSGLIGCMSFGVKSLLTPDKEISGWYYLLGEHLGRTKHLKVARRRLRPLRDTNRRKPRGKDTEKRNKRKITIPRGKDGFGFTICCDSPVRVQAVDSGGPAERAGLQQLDTVLQLNERPVEHWKCVELAHEIRSCPSEIILLVWRMVPQVKPGPDGGVLRRASCKSTHDLQSPPNKREKNCTHGVQARPEQRHSCHLVCDSSDGLLLGGWERYTEVAKRGGQHTLPALSRATAPTDPNYIILAPLNPGSQLLRPVYQEDTIPEESGSPSKGKSYTGLGKKSRLMKTVQTMKGHGNYQNCPVVRPHATHSSYGTYVTLAPKVLVFPVFVQPLDLCNPARTLLLSEELLLYEGRNKAAEVTLFAYSDLLLFTKEDEPGRCDVLRNPLYLQSVKLQEGSSEDLKFCVLYLAEKAECLFTLEAHSQEQKKRVCWCLSENIAKQQQLAASPPDSKKLHTDADHKKEMAPDKANKPGAEDSPPSKEPSPGQELPPGQDLPPNKDSPSGQEPAPSQEPLSSKDSATSEGSPPGPDAPPSKDVPPCQEPPPAQDLSPCQDLPAGQEPLPHQDPLLTKDLPAIQESPTRDLPPCQDLPPSQVSLPAKALTEDTMSSGDLLAATGDPPAAPRPAFVIPEVRLDSTYSQKAGAEQGCSGDEEDAEEAEEVEEGEEGEEDEDEDTSDDNYGERSEAKRSSMIETGQGAEGGLSLRVQNSLRRRTHSEGSLLQEPRGPCFASDTTLHCSDGEGAASTWGMPSPSTLKKELGRNGGSMHHLSLFFTGHRKMSGADTVGDDDEASRKRKSKNRAKDMKNKLGIFRRRNESPGAPPAGKADKMMKSFRPTSEEALKWGESLEKLLVHKDGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPP

SEQ ID NO: 12 - protein, 575 aa, Homo sapiens
GQLRLSIDAQDRVLLLHVIEGKGLISKQPGTCDPYVKISLIPEDSRLRHQKTQTVPDCRDPAFHEHFFCPVQEEDDQKRLLVTVWNRASQSRQSGLIGCMSFGVKSLLTPDKEISGWYYLLGEHLGRTKHLKVARRRLRPLRDPLLRMPGGGDTENGKKLKITIPRGKDGFGFTICCDSPVRVQAVDSGGPAERAGLQQLDTVLQLNERPVEHWKCVELAHEIRSCPSEIILLVWRMVPQVKPGPDGGVLRRASCKSTHDLQSPPNKREKNCTHGVQARPEQRHSCHLVCDSSDGLLLGGWERYTEVAKRGGQHTLPALSRATAPTDPNYIILAPLNPGSQLLRPVYQEDTIPEESGSPSKGKSYTGLGKKSRLMKTVQTMKGHGNYQNCPVVRPHATHSSYGTYVTLAPKVLVFPVFVQPLDLCNPARTLLLSEELLLYEGRNKAAEVTLFAYSDLLLFTKEDEPGRCDVLRNPLYLQSVKLQEGSSEDLKFCVLYLAEKAECLFTLEAHSQEQKKRVCWCLSENIAKQQQLAASPPDSKKLHPFGSLQQEMGPVNSTNATQDRSFTSPGQTLI

SEQ ID NO: 13 - protein, 916 aa, Homo sapiens
MNRFNGLCKVCSERRYRQITIPRGKDGFGFTICCDSPVRVQAVDSGGPAERAGLQQLDTVLQLNERPVEHWKCVELAHEIRSCPSEIILLVWRMVPQVKPGPDGGVLRRASCKSTHDLQSPPNKREKNCTHGVQARPEQRHSCHLVCDSSDGLLLGGWERYTEVAKRGGQHTLPALSRATAPTDPNYIILAPLNPGSQLLRPVYQEDTIPEESGSPSKGKSYTGLGKKSRLMKTVQTMKGHGNYQNCPVVRPHATHSSYGTYVTLAPKVLVFPVFVQPLDLCNPARTLLLSEELLLYEGRNKAAEVTLFAYSDLLLFTKEDEPGRCDVLRNPLYLQSVKLQEGSSEDLKFCVLYLAEKAECLFTLEAHSQEQKKRVCWCLSENIAKQQQLAASPPDSKMFETEADEKREMALEEGKGPGAEDSPPSKEPSPGQELPPGQDLPPNKDSPSGQEPAPSQEPLSSKDSATSEGSPPGPDAPPSKDVPPCQEPPPAQDLSPCQDLPAGQEPLPHQDPLLTKDLPAIQESPTRDLPPCQDLPPSQVSLPAKALTEDTMSSGDLLAATGDPPAAPRPAFVIPEVRLDSTYSQKAGAEQGCSGDEEDAEEAEEVEEGEEGEEDEDEDTSDDNYGERSEAKRSSMIETGQGAEGGLSLRVQNSLRRRTHSEGSLLQEPRGPCFASDTTLHCSDGEGAASTWGMPSPSTLKKELGRNGGSMHHLSLFFTGHRKMSGADTVGDDDEASRKRKSKNRAKDMKNKLGIFRRRNESPGAPPAGKADKMMKSFRPTSEEALKWGESLEKLLVHKDGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPP

SEQ ID NO: 14 - protein, 63 aa, Drosophila melanogaster
WKTNKSESQYTSLEYDFRVTCDLNYYGSGCAKFCRPRDDSFGHSTCSETGEIICLTGWQGDYC

SEQ ID NO: 15 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 16 - protein, 63 aa, Mus sp.
WSQDLHSSGRTDLRYSYRFVCDEHYYGEGCSVFCRPRDDAFGHFTCGDRGEKMCDPGWKGQYC

SEQ ID NO: 17 - protein, 63 aa, Rattus rattus
WSQDLHSSGRTDLRYSYRFVCDEHYYGEGCSVFCRPRDDAFGHFTCGERGEKMCDPGWKGQYC

SEQ ID NO: 18 - protein, 63 aa, Mus sp.
WRTDEQNDTLTRLSYSYRVICSDNYYGESCSRLCKKRDDHFGHYECQPDGSLSCLPGWTGKYC

SEQ ID NO: 19 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 20 - protein, 63 aa, Rattus rattus
WQTLKQNTGIAHFEYQIRVTCDDHYYGFGCNKFCRPRDDFFGHYACDQNGNKTCMEGWMGPEC

SEQ ID NO: 21 - protein, 63 aa, Mus sp.
WQTLKQNTGIAHFEYQIRVTCDDHYYGFGCNKFCRPRDDFFGHYACDQNGNKTCMEGWMGPDC

SEQ ID NO: 22 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 23 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 24 - protein, 63 aa, Gallus sp.
WKTLQFNGPVANFEVQIRVKCDENYYSALCNKFCGPRDDFVGHYTCDQNGNKACMEGWMGEEC

SEQ ID NO: 25 - protein, 63 aa, Mus sp.
WKSLHFSGHVAHLELQIRVRCDENYYSATCNKFCRPRNDFFGHYTCDQYGNKACMDGWMGKEC

SEQ ID NO: 26 - protein, 63 aa, Homo sapiens; identical to SEQ ID NO: 25

SEQ ID NO: 27 - protein, 63 aa, Rattus rattus; identical to SEQ ID NO: 25

SEQ ID NO: 28 - protein, 63 aa, Homo sapiens; identical to SEQ ID NO: 25

SEQ ID NO: 29 - protein, 63 aa, Drosophila melanogaster
WKTLDHIGRNARITYRVRVQCAVTYYNTTCTTFCRPRDDQFGHYACGSEGQKLCLNGWQGVNC

SEQ ID NO: 30 - protein, 723 aa, Homo sapiens [sequence omitted]

SEQ ID NO: 31 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 32 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 33 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 34 - protein, 1238 aa, Homo sapiens [sequence omitted]

SEQ ID NO: 35 - DNA, 30 bp, Artificial
  PCR primer
tttgaattccggcggcagcatggccagctc

SEQ ID NO: 36 - DNA, 33 bp, Artificial
  PCR primer
tttggatcctcgaaccggaacttcttggtctcc

SEQ ID NO: 37 - DNA, 17 bp, Artificial
  PCR primer
tcatcggaagagagtag

SEQ ID NO: 38 - DNA, 22 bp, Artificial
  PCR primer
cgttttaaaacctaagagtcac

SEQ ID NO: 39 - DNA, 32 bp, Artificial
  PCR prmer
ctattcgatgatgaagataccccaccaaaccc

SEQ ID NO: 40 - DNA, 32 bp, Artificial
  PCR primer
aagtgaacttgcggggtttttcagtatctacg

SEQ ID NO: 41 - DNA, 20 bp, Artificial
  PCR primer
ttacccatacgatgttccag

SEQ ID NO: 42 - DNA, 21 bp, Artificial
  Sequencing Primer
gtgcgacatcatcatcggaag

SEQ ID NO: 43 - DNA, 21 bp, Artificial
  Sequencing primer
cctaagagtcactttaaaatt

SEQ ID NO: 44 - DNA, 20 bp, Artificial
  Sequencing primer
gccccagtgagatcatccta

SEQ ID NO: 45 - DNA, 20 bp, Artificial
  Sequencing primer
ctgcaagtcgacacatgacc

SEQ ID NO: 46 - DNA, 20 bp, Artificial
  Sequencing primer
ggataccatccccgaagaat

SEQ ID NO: 47 - DNA, 20 bp, Artificial
  Sequencing primer
tattcggacctgctgctctt

SEQ ID NO: 48 - DNA, 20 bp, Artificial
  Sequencing primer
gaggaggaggaggacattcc

SEQ ID NO: 49 - DNA, 20 bp, Artificial
  Sequencing primer
cagcagaaagtcccaagagg

SEQ ID NO: 50 - protein, 864 aa, Artificial
  Expressed peptide sequence
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 43 aa, artificial sequence
  MISC_FEATURE (2)..(9): X indicates any amino acid
CXXXXXXXXCXXXCXXXXXXXXXXXCXXXXXXXCXXXXXXXXC

SEQ ID NO: 52 - protein, 43 aa, artificial sequence; identical to SEQ ID NO: 51
  MISC_FEATURE (2)..(4): X is any amino acid

SEQ ID NO: 53 - protein, 43 aa, artificial sequence
  MISC_FEATURE (2)..(4): X is any amino acid
CXXXYYXXXCXXXCRPRXDXFGHXXCXXXGXXXCXXGWXGXXC

SEQ ID NO: 54 - protein, 18 aa, artificial sequence
  MISC_FEATURE (1)..(1): X is any amino acid
XCXCXCXCXCXGXXGXCX